(19) United States
(12) Patent Application Publication
(10) Pub. No.: US 2013/0030462 A1
(43) Pub. Date: Jan. 31, 2013

(54) LAPAROSCOPIC SURGICAL DEVICE

(71) Applicants: Ronan Keating, Galway (IE); Gerard Rabbitte, Galway (IE); Barry Russell, Galway (IE)

(72) Inventors: Ronan Keating, Galway (IE); Gerard Rabbitte, Galway (IE); Barry Russell, Galway (IE)

(73) Assignee: neoSurgical Limited, Galway, IE (unknown)

(21) Appl. No.: 13/517,479

(22) PCT Filed: Dec. 21, 2010
(86) PCT No.: PCT/EP10/70398

§ 371 Date: Jun. 20, 2012

(30) Foreign Application Priority Data
Dec. 22, 2009 (GB) 09223298

(51) Int. Cl. A61B 17/29

(52) U.S. Cl. 606/206

(57) ABSTRACT

A laparoscopic surgical device comprising a pair of jaws (115, 116) defining a mouth within which at least a portion of an organ or tissue may be grasped is described. The jaws are biased towards one another so as to normally adopt a closed configuration, the device being dimensioned to be operably passed fully through a trocar into the abdominal cavity wherein it may be manipulated by a surgeon or other operator to grasp the desired target organ or tissue.

### FIELD OF THE INVENTION

The present invention relates to traditional laparoscopic surgery and the emerging technique referred to as single incision laparoscopy. The invention more particularly relates to devices, preferably retraction devices, for use in laparoscopic surgery and single incision surgery. The invention also relates to method of laparoscopic surgery and single incision surgery.

### BACKGROUND

Laparoscopic surgery which is also known as keyhole surgery or minimally invasive surgery (MIS) is a surgical technique in which operations in the abdomen are performed through small incisions provided in the abdomen wall. The incisions are typically of the order of 0.5-2.5 cm and provide the surgeon with access to the interior cavity for performing the necessary surgical operation. The cavity is typically inflated with carbon dioxide, to increase the volume of the cavity so as to provide the necessary working and viewing space for the surgeon.

The surgical instruments are inserted into the abdominal cavity through a cannula or trocar located within the incisions in the cavity wall. For laparoscopic surgery, each operation typically requires a minimum number of such incisions to enable the use of a sufficient number of instruments as determined by the surgeon. The control of the instruments is effected outside the body cavity. By using such minimally invasive surgical techniques as opposed to earlier open surgical procedures there are a number of advantages including the fact that the smaller incisions used amongst other factors reduces the pain of the operation and shortens recovery times for the patients. There are many factors contributing to reduced patient morbidities with minimally invasive surgery over open surgery which make this an increasingly attractive option for patient and surgeon. For this reason there is a more recent push towards even less invasive laparoscopic approaches. There are various names and acronyms attached to this emerging technique of surgery including, Single Incision Laparoscopic Surgery (SILS), Lapro-Endoscopic Single-Site Surgery (LESS) amongst many others. Fundamentally, the aim is to operate from one site, typically at the umbilicus, thereby eliminating the extra ports and improving cosmesis for the patient. However, this approach introduces additional constraints on the operator over the traditional laparoscopic approach and is likely to require new flexible and accessible instruments to complete the surgical procedure efficiently.

As the surgery is completed through a relatively small number of small diameter access points to the interior cavity, it is preferable to have only one operator of the instrumentation within the cavity. However due to the number of instruments that may need to be operated concurrently, there is often a requirement for two or more persons to operate the instruments concurrently, which can be a nuisance in that each of the multiple persons add cost and potentially increases the risk of a complication occurring. The addition of extra personnel is a challenge for private practices that may not have the human resources to meet this need. Whether in a public or private hospital setting, the majority of surgeons would prefer to be in control of their set up and the operating environment.

Furthermore, the site of the surgery is often occluded by another organ that needs to be moved out of the field of view to allow access to the surgery site. In the context of traditional open surgery where access to the operating site is more open, this can be easily achieved by the surgeons hand or an assistants hand or a simple retraction device held in place by the operator's hand. This is often considered a conventional step in the operation. However with laparoscopic surgery, while the moving of occluding organs is still necessary, it is more difficult to achieve and has typically been achieved in one of two ways, both of which utilise retraction devices.

Known retraction devices work on the principle of holding up the target organ from outside the abdominal cavity. They are a mix of single patient use (SPU) and reusable devices depending on the manufacturer. Typically, a metal shaft is inserted via a 10/12 mm or 5 mm port and has various applicator end section designs depending on the manufacturer and model. These can for example be dimensioned to resemble finger type designs, a simple wedge shape or toothed jaws. Some of these designs are of metal construct but there are a number of inflatable types also available. These end applicator sections are designed and constructed in many different ways but essentially they all perform a similar function in lifting the target organ. This target organ will depend on the actual operation being completed but in the context of surgery in the area of the gastroesophageal junction and surrounding structures the left lobe of the liver is typically required to be lifted out of the field of view. They are advanced under the target organ, for example the liver, and the liver is then leveraged up and out of the field of view using a rigid lever. The device is then held in position by an assistant or some devices are fixed to an external support frame which acts as an aid to fix it into position. In all arrangements the retraction device is secured from the outside and most designs require a dedicated port throughout usage.

Another common retraction method is applied to the right side of the liver. Typically this method is used to grasp, retract and orientate the gallbladder and the attached right liver lobe in to a position that provides a ‘critical view’ of the key structures at the root of the gallbladder. This procedure is known as the Laparoscopic Cholecystectomy. The typical set up for this procedure requires 4 ports, one of which is dedicated to the retraction of the gallbladder and right liver lobe.

The use of dedicated ports suffers in that an additional incision is required, and as will be appreciated from above, there is a desire in laparoscopic surgery to keep the number of incisions to a minimum. There is also a cost disadvantage of having to employ an additional port. Furthermore the maintaining of the retraction device in situ using a person requires that person to maintain a static hold for the entire procedure or certainly over prolonged periods of time causing fatigue. Fatigue usually leads to movement and in most cases there is a lack of operator control from the outset as they are relying on an assistant. Other device types require the assembly of an external fixation scaffolding around the operating table so as they can be fixed to it for the duration of the procedure and this can occupy valuable space and hinder the surgeon in his performance of the surgery. They are also reusable and require sterilisation and maintenance.

Therefore there are a number of problems associated with existing liver retraction devices and their methods of use. There is also a distinct shortage of solutions to deal with emerging techniques such as the single incision surgery and all of its associated procedures including but not limited to Laparoscopic Cholecystectomy, Laparoscopic Gastric Banding and Bypass, and Laparoscopic Nissen Fundoplication. Traditional laparoscopic approaches also offer challenging retraction in operations such as, but not restricted to, laparoscopic colon procedures. During this procedure the small bowel typically has to be maintained/retracted in a position out of the field of view of the target large bowel or colon. Therefore both approaches, namely, traditional laparoscopic and single incision surgeries, offer many retraction difficulties for the operator/surgeon. The emergence of the single incision approach leads to increased difficulties as there are even more limiting factors due to the position of the single incision and the operating difficulties this presents to the surgeon.

### SUMMARY

These and other problems are addressed in accordance with the present teaching by provision of a laparoscopic surgical device. There is also provided a method for providing access to surgical sites within the abdomen that would otherwise be occluded by other organs.

In a first arrangement a surgical device is provided having a pair of jaws that are biased towards one another so as to normally adopt a closed configuration. The device in such a form may be considered a grasper and may comprise a spring or other biasing means configured to provide the necessary force to effect an operable biasing of the jaws towards one another. In certain configurations a head portion may be provided comprising the jaws, in other configurations the head portion may be omitted.

The grasper is dimensioned to be operably passed fully through a trocar into the abdominal cavity wherein it may be manipulated by a surgeon or other operator to effect a hold on a desired target organ. On location of the grasper relative to the organ and on retention of at least a portion of the organ within the jaws of the grasper, movement of the grasper will effect a corresponding movement of the retained organ. On reaching a desired location, the grasper can be secured to maintain this position of the moved or retracted organ so as to facilitate surgery. Once the surgery has been completed a release of the organ from the jaws of the grasper will allow the organ to return to its normal position within the abdominal cavity.

At least one of the jaws is desirably pivotable relative to the other of the jaws to allow for movement of the jaws away from one another to facilitate the presentation of an organ into a mouth region of the grasper. As the jaws are naturally biased towards one another, this pivoting requires provision of an external force so as to effect a separation of the jaws away from one another. The grasper may include a pivot pin coupling the jaws to one another. The pin is desirably mounted so as to be substantially perpendicular to a longitudinal axis of the head. The pivot pin provides a fulcrum such that application of a force to a first side of the pin effects a corresponding movement of the jaws on a second opposing side of the pin. In another arrangement, not shown, the upper and lower jaws may also interface like a rocker and not require a pivot pin as a compression spring holds the two together.

In a first arrangement the force is applied to a first jaw to effect a movement of that first jaw away from the second jaw. In a second arrangement the force is applied to each of the first and second jaws such that the separation of the jaws from one another is effected by a relative movement of each of the first and second jaws.

The biasing of the jaws may be provided by a compression spring located between the pivot pin and the mouth of grasper head, i.e. proximal to the mouth of the grasper. The jaws may contain teeth. Desirably such teeth, if provided, are located in the mouth region of the jaws such that on presentation of the grasper to the organ, the teeth will engage with and effect a retention of the organ within the mouth of the grasper. In a first arrangement each of the first and second jaws have teeth. The teeth of the first and second jaws are desirably configured such that on bringing the first and second jaws towards one another, the teeth of the first jaw will inter-engage with the teeth of the second jaw so as to allow a complete closing of the mouth of the grasper.

In another arrangement, one of the two jaws comprises teeth and the second of the two jaws provides a planar surface. In yet another arrangement, a first jaw comprises first and second sets of teeth that extend alongside each other but are offset from one another.

At least one of the jaws may comprise a tapered lead-in portion, tapering from a distal portion of the grasper head inwardly towards the mouth region. In this way, in a closed orientation, the jaws are separated from one another at the lead-in portion. The lead-in portion may comprise a set of teeth of finer grade to that of the teeth located within the mouth portion of the grasper. The teeth may be provided traumatic or atraumatic depending on the retraction application or the value of the organ/tissue being grasped by the grasper.

The grasper may further comprise a body portion coupled or integrally formed with the head portion. The body portion, if provided, desirably provides a coupling contact for effecting a securing of the grasper at a desired position within the abdominal cavity.

In a first arrangement the body portion extends longitudinally away from the head and has a length substantially greater than the length of the head. In such an arrangement, the body portion may comprise a plurality of coupling surfaces spaced apart along the body portion and configured to cooperation with a separate coupling tool. In a first arrangement the coupling surfaces are provided as a plurality of individual coupling ports extending through the body portion. Each of the coupling ports desirably comprises a tapered mouth or chamfered entrance to assist the presentation and location of the separate coupling tool within the coupling port. In such an arrangement, movement of the coupling tool from a first coupling port to a second coupling port requires a complete separation of the coupling tool from a first coupling port of the grasper and the re-presentation of the coupling tool to a second coupling port of the tool. In a second arrangement a plurality of inter-connected slots are provided along the length of the body, each of the slots providing a coupling surface for cooperation with the separate coupling tool. In such an arrangement a change in the orientation of the coupling tool allows it to move from a first orientation to a second orientation. This change in orientation allows for the coupling tool to be slid within the body portion and moved from one slot to another. In yet a further arrangement the body portion comprises only one coupling surface which again is configured for co-operation with a separate coupling tool. Such a latter arrangement is typically used where the intended ultimate location of the grasper within the abdominal cavity is well defined and the surgeon does not require the same level of flexibility as is provided by having a plurality of coupling surfaces.

The body may be configured to be moveable relative to the head of the grasper. In a first arrangement the body may be moveable along the longitudinal axis of the grasper. Such an arrangement may allow for a compression of the body towards the head so as to effect a reduction in length of the grasper. Such an arrangement is advantageous in circumstances where the grasper is to be secured against a surface. By spring loading the body portion relative to the head, a movement of the body towards the head can be used to reduce the length of the grasper. This allows a location of the grasper within the abdominal cavity. The release of the biasing force that effects a compression of the body will allow the grasper to attempt to return to its normal elongated state. If the available space is not sufficient the grasper will be retained in position by being positively biased against the surface preventing the extension to its normal position. The body portion may be coupled to a surface engagement portion which provides a contact surface for engaging against the abdominal cavity surface. The surface engagement portion may comprise a flexible portion which may be contracted to reduce its dimensions but which on deployment within the abdominal cavity may be extended to provide a substantially enlarged contact surface for contacting against the abdominal cavity surface.

The surface engagement portion may be moveable relative to the head portion of the grasper such that it may be angularly orientated relative to the head portion. This may be achieved by having the surface engagement portion moveable to both the body portion and the head portion or simply having the surface engagement portion moveable relative to the head portion. The surface engagement portion may also feature an adhesive or textured surface to enable improved attachment. In a first arrangement the surface engagement portion may be rotated relative to the head portion. In a second arrangement the surface engagement portion may be moved so as to be offset from the longitudinal axis of the grasper. In a third arrangement the surface engagement portion may be configured to be both rotatable and angularly offset from the longitudinal axis of the grasper.

The body portion may be segmented to comprise a head proximal body portion and a head distal body portion. The first and second segments of the body portion may be moveable relative to one another. In this way a change in orientation of the first segment relative to the second segment may be effected.

In another arrangement the body portion is coupled to a suture which operably may be used to tether the grasper so as to secure a retained organ/tissue at a suitable location. Use of such an arrangement typically requires a passing of the suture through the abdominal wall.

In a further arrangement, the body portion comprises a deployable needle so as to allow a fixing of the grasper to an inner abdominal wall or other desired feature. In a first arrangement the needle is normally sheathed or otherwise protected to ensure inadvertent contact of the needle with organs/tissue within the abdominal cavity is not achieved. Such a sheathing may be provided by locating the needle within a spring loaded needle protector. Such a protector may include one or more grips to prevent it slipping relative to the abdominal wall. On application of a load to the protector, the protector is configured to retract thereby exposing the needle and allowing for a puncturing of tissue and an anchoring of the grasper.

In another arrangement the needle is mounted relative to the body portion so as to be pivotable from a first position wherein the needle is located within the extremities of the body portion to a second position wherein the needle is moveable away from the body portion and is angularly positioned relative to the body portion. On adoption of the second position the needle may be presented to the inner abdominal wall or other feature within the cavity to effect the necessary securing of the grasper.

In yet a further arrangement the device defines a clasping element again comprising a first and a second jaw that are biased towards one another. In this arrangement the jaws are integral with one another and are fabricated in a shape memory material which may be copper-based, NiTi (nickel and titanium)-based, or polymer-based materials. By fabricating each of the first and second jaws in such a memory material which may deform but which will return to its normal state the jaws can be configured through a suitable shaping of the jaws to be naturally biased towards one another. In this way the jaws may be separated to allow for the location of an organ or other tissue therebetween. The construct of the jaws is such that jaws will tend to move towards one another, the movement effecting a capture or clasping of the organ/tissue therebetween. A plastic or elastic material may also be used to fabricate this arrangement.

Such a clasping element may be used in combination with a suture or other fastening means to effect a movement of the clasped organ or tissue from its normal resting position to an operational site where for example access to a site normally occluded by the normal resting position is required.

Accordingly there is provided a surgical device as detailed in the independent claims. Advantageous features are provided in the dependent claims. A tool and method as detailed in independent claims are also provided.

These and other features of the present invention will now be described with reference to an exemplary arrangement thereof which is provided to assist in an understanding of the teaching of the invention but is not intended to be construed as limiting the invention to the exemplary arrangements which follow.

### BRIEF DESCRIPTION OF THE DRAWINGS

The present invention will now be described with reference to the accompanying drawings in which:

FIG. 1A is a perspective of a grasper in accordance with the present teaching.

FIG. 1B is a perspective view of an alternative grasper in accordance with the present teaching.

FIGS. 1C to 1F are sectional views through alternative grasper in accordance with the present teaching.

FIGS. 1G through 1I show the grasper of FIG. 1C in various states of jaw opening.

FIG. 1J is a side view, FIG. 1K a view from above and FIG. 1L a sectional view of another grasper configuration.

FIG. 2A is a side view, FIG. 2B a view from above and FIG. 2C a sectional view of the grasper of FIG. 1.

FIG. 2D is a sectional view and FIG. 2E a perspective view of an alternative grasper.

FIG. 2F is a side view, FIG. 2G a view from above and FIG. 2H a sectional view of a two-headed grasper.

FIG. 3A shows various coupling mechanisms that could be used for securing a spring within the body.

FIG. 3B is a detailed view of a head of a grasper in accordance with the present teaching.

FIG. 3C is a side view of the grasper of FIG. 1 with an open mouth.

FIG. 4A is a side view with closed jaws and FIG. 4B a side view with open jaws of an alternative grasper in accordance with the present teaching.

FIG. 5A is a side view and FIG. 5B a perspective view from above of an alternative grasper in accordance with the present teaching.

FIG. 6A is a side view and FIG. 6B a perspective view from above of an alternative grasper in accordance with the present teaching.

FIG. 7A is a side view, FIG. 7B a view from above and FIG. 7C a sectional view of an alternative grasper.

FIG. 8 is a perspective view of the grasper of FIG. 7.

FIG. 9A is a side view, FIG. 9B a view from above, and FIG. 9C is a sectional view of an alternative grasper.

FIG. 10 is a perspective view of an alternative grasper.

FIG. 11A is side view and FIG. 11B a perspective view of another grasper in accordance with the present teaching.

FIG. 12A is a side view and FIG. 12B a top view of another grasper in accordance with the present teaching.

FIG. 12C is a perspective view of the grasper of FIGS. 12A and 12B.

FIG. 12D is a side view, FIG. 12E a view from above, FIG. 12F a sectional view of an alternative needle arrangement in accordance with the present teaching.

FIG. 12G is a perspective view of an alternative grasper coupling element.

FIG. 13A is a side view, FIG. 13B a view from above, FIG. 13C a sectional view and FIG. 13D a perspective view of an alternative grasper in accordance with the present teaching.

FIGS. 14A and 14B are details of alternative mounting arrangements for the needle of FIG. 13.

FIG. 15A is a side view, FIG. 15B a view from above, FIG. 15C a sectional view and FIG. 15D a perspective view of the grasper of FIG. 13 with the needle in a deployed state.

FIG. 16 shows an alternative configuration for a grasper in accordance with the present teaching.

FIG. 17 shows a coupling arrangement for coupling the grasper of the present teaching to an external tool.

FIG. 18 shows an alternative coupling arrangement for coupling the grasper of the present teaching to an external tool.

FIG. 19 shows a microtrocar in accordance with the present teaching coupled to a grasper of FIG. 1.

FIGS. 19A and 19B show details of alternative tip portions of the needle section of a microtrocar such as that detailed in FIG. 19.

FIGS. 19C, 19D and 19E show alternative atraumatic tip configurations of a microtrocar such as that detailed in FIG. 19.

FIG. 19F is a plan view of use of a microtrocar.

FIG. 19G is a sectional view of the microtrocar in use and FIG. 19H is a perspective view of the same microtrocar.

FIG. 19I is a perspective view showing another configuration that may be used for coupling a grasper to a microtrocar in accordance with the present teaching.

FIG. 20 is a schematic showing use of a plurality of graspers in accordance with the present teaching within an abdominal cavity.

FIG. 21 shows various configurations for a clasping device in accordance with the present teaching.

FIG. 22A is a sectional view through another configuration of a grasper in accordance with the present teaching.

FIG. 22B is a perspective view of the grasper of FIG. 22A.

FIG. 23A is a top view of another configuration of a grasper in accordance with the present teaching.

FIG. 23B is a sectional view of the grasper of FIG. 23A.

FIG. 23C is a detail of a portion of the grasper of FIG. 23a and FIG. 23B.

FIG. 24A is a sectional view through another configuration of a grasper in accordance with the present teaching.

FIG. 24B is a perspective view of the grasper of FIG. 24A.

FIG. 25A is a sectional view through another configuration of a grasper in accordance with the present teaching coupled to an external actuator.

FIG. 25B is a sectional view of the grasper of FIG. 25A with the external actuator angularly offset from the grasper.

FIG. 25C is a side view of the grasper and external actuator of FIG. 25B, showing the external actuator in more detail

FIG. 25D is a side view of the grasper and external actuator of FIG. 25A, showing the external actuator in more detail

FIG. 25E is a detail of the driver of the external actuator.

### DETAILED DESCRIPTION OF THE DRAWINGS

Exemplary arrangements of devices for laparoscopic surgery will now be described to assist in an understanding of the present teaching.

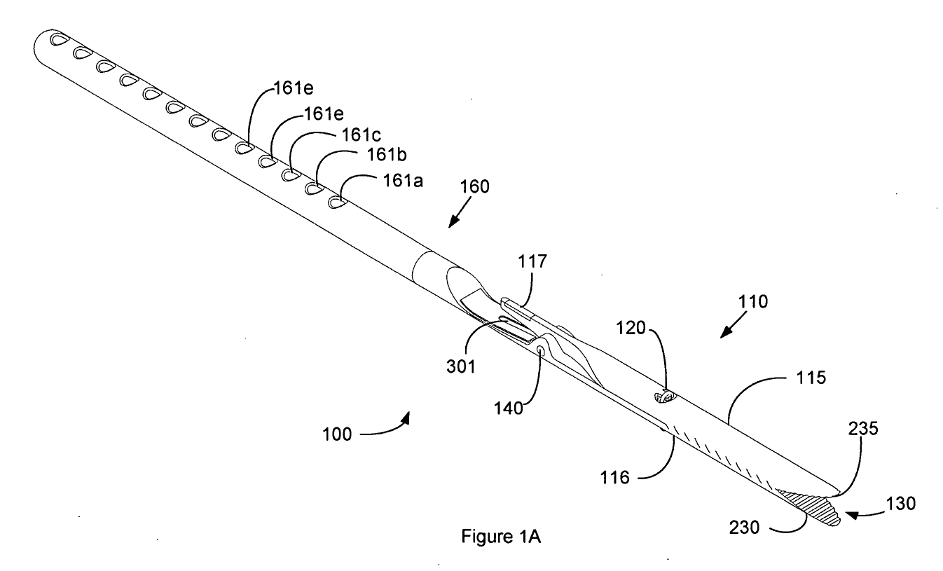

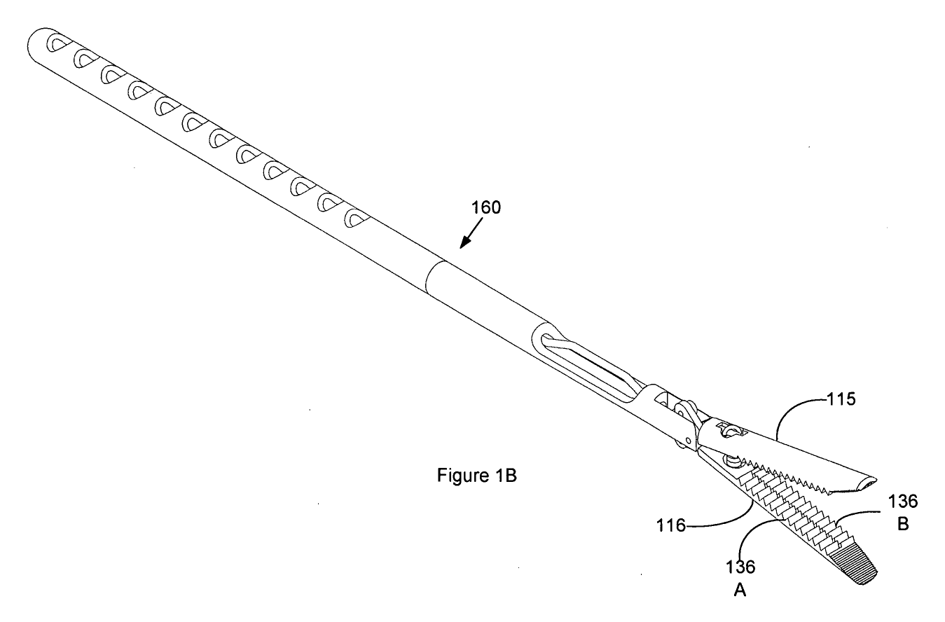

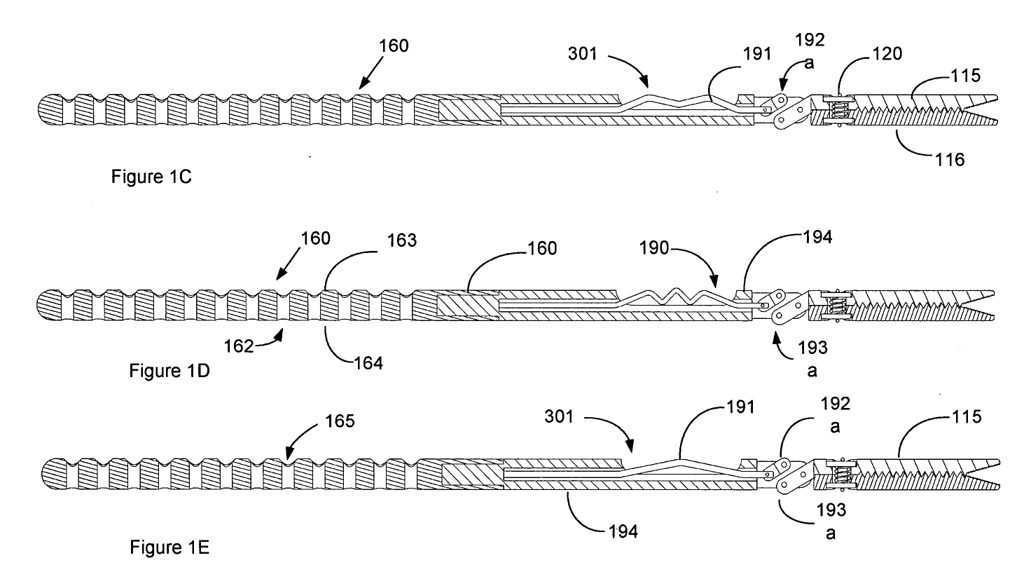

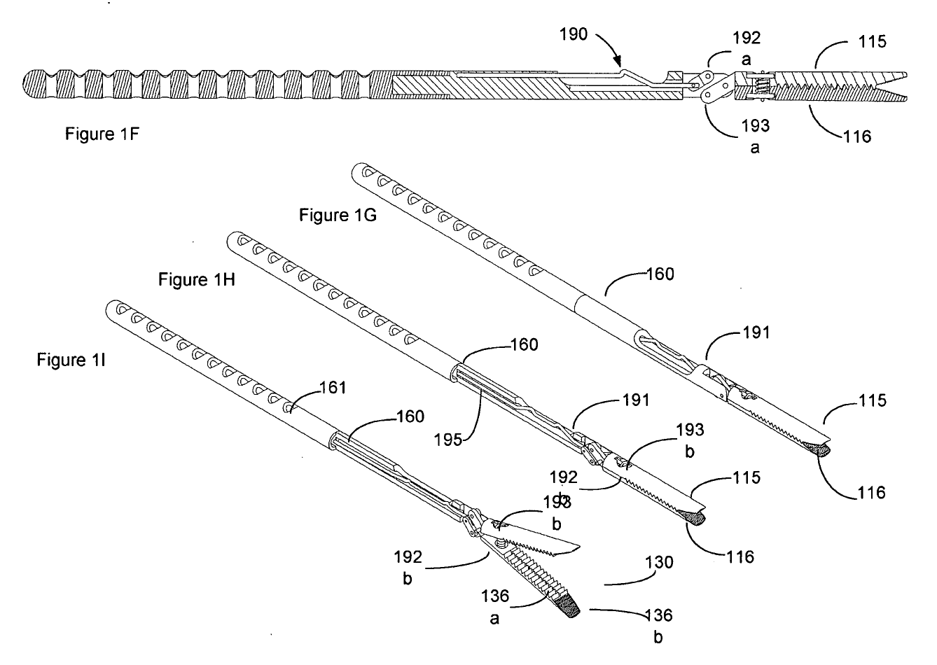

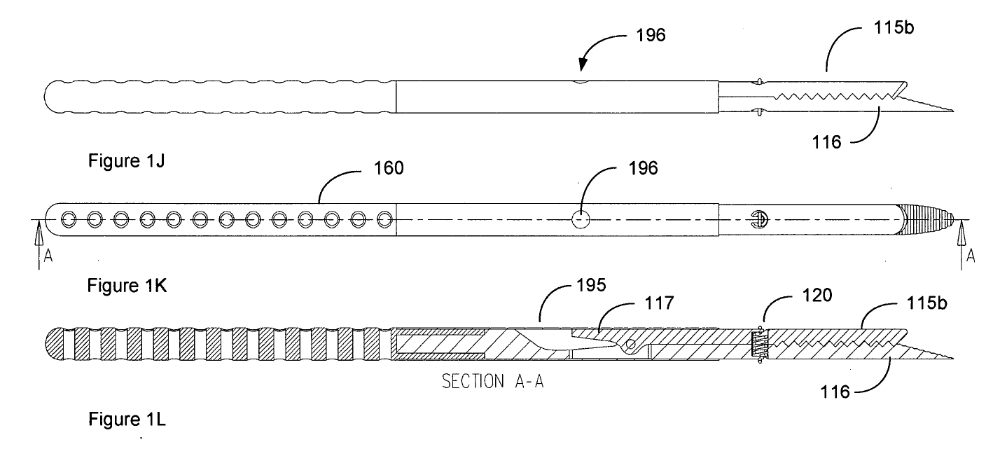

FIG. 1 shows an exemplary surgical device in the form of a surgical grasper 100 having a head portion 110 with a pair of jaws 115, 116 that are biased towards one another so as to normally adopt a closed configuration. The jaws define a mouth 130 therebetween with which at least a portion of an organ or tissue may be presented and grasped. The grasper comprises a spring 120 or other biasing means configured to provide the necessary force to effect an operable biasing of the jaws 115, 116 towards one another so as to operably retain the organ within the mouth 130 of the grasper. The spring may be manufactured from a variety of metals such as Stainless Steel or Nitinol. Nitinol would be particularly advantageous as its linear elastic properties would facilitate a larger opening of the jaw. Springs may also be placed in series to provide a larger compressive force within the restricted dimensions of the device.

The grasper is dimensioned to be operably passed fully through a trocar into the abdominal cavity wherein it may be manipulated by a surgeon or other operator to effect a hold on a desired target organ. These dimensions will depend on the dimensions of the trocar with which the grasper is to be used. For example if intended use is with a 5 mm trocar the grasper should have a maximum outside diameter of 5 mm. If use is with a 10 mm trocar then maximum outside diameter should be 10 mm.

On location of the grasper relative to the organ and a retention of at least a portion of the organ within the mouth 130 defined between the jaws 115, 116 of the grasper, movement of the grasper will effect a corresponding movement of the retained organ. On reaching a desired location, the grasper can be secured to maintain this position of the moved or retracted organ so as to facilitate surgery. Once the surgery has been completed a release of the organ from the jaws of the grasper will allow the organ to return to its normal position within the abdominal cavity.

At least one of the jaws is desirably pivotable relative to the other to allow for movement of the jaws away from one another to facilitate the presentation of an organ into a mouth region of the grasper. In the arrangement of FIG. 1 the first 115 or upper jaw is pivotable relative to the second or lower jaw 116. As the jaws are naturally biased towards one another, this pivoting requires provision of an external force so as to effect a separation of the jaws away from one another. To achieve this pivoting, the grasper in this exemplary arrangement, includes a pivot pin 140 coupling the jaws 115, 116 to one another. The pin is desirably mounted so as to be substantially perpendicular to the longitudinal axis of the head. The pivot pin provides a fulcrum such that application of a force to a first side of the pin effects a corresponding movement of the jaws on a second opposing side of the pin.

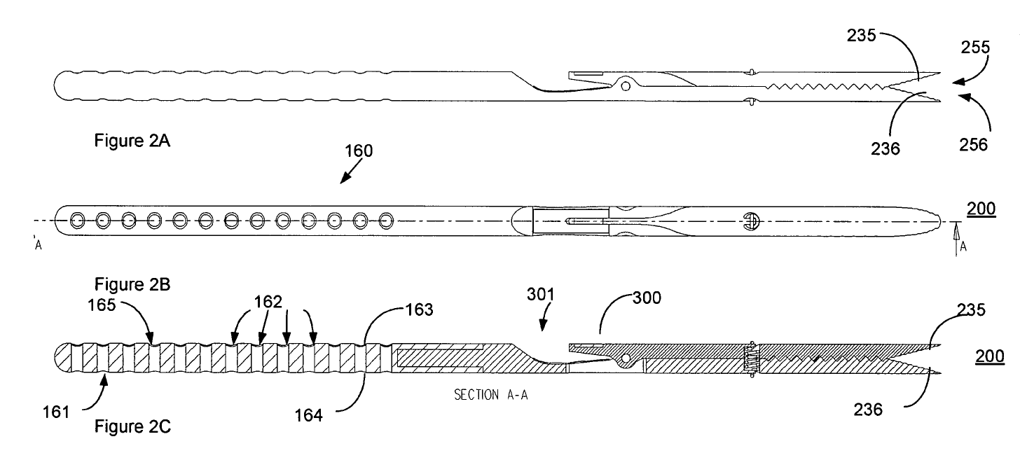

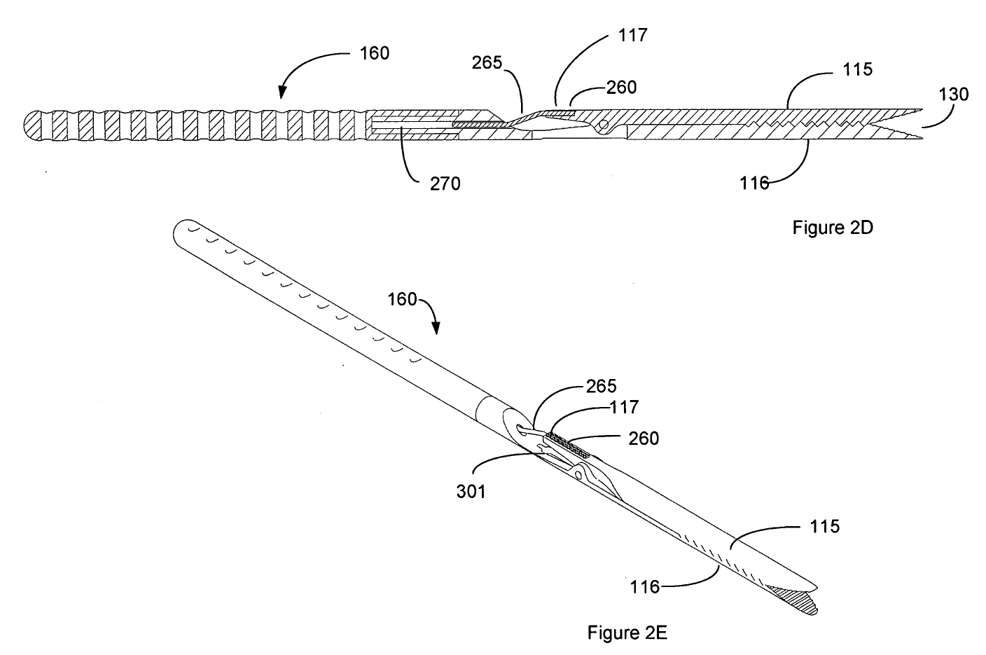

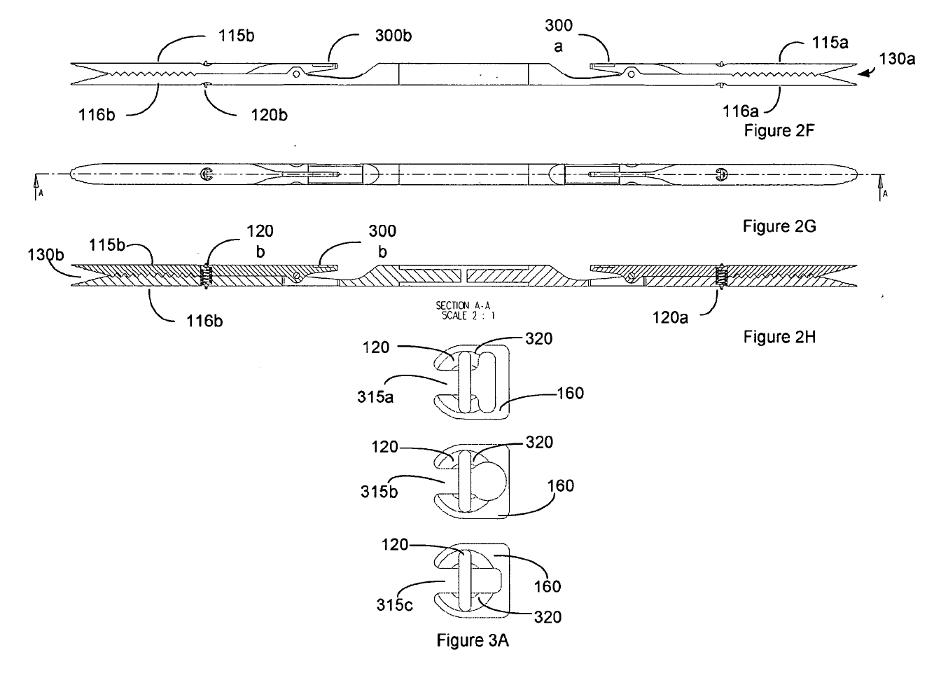

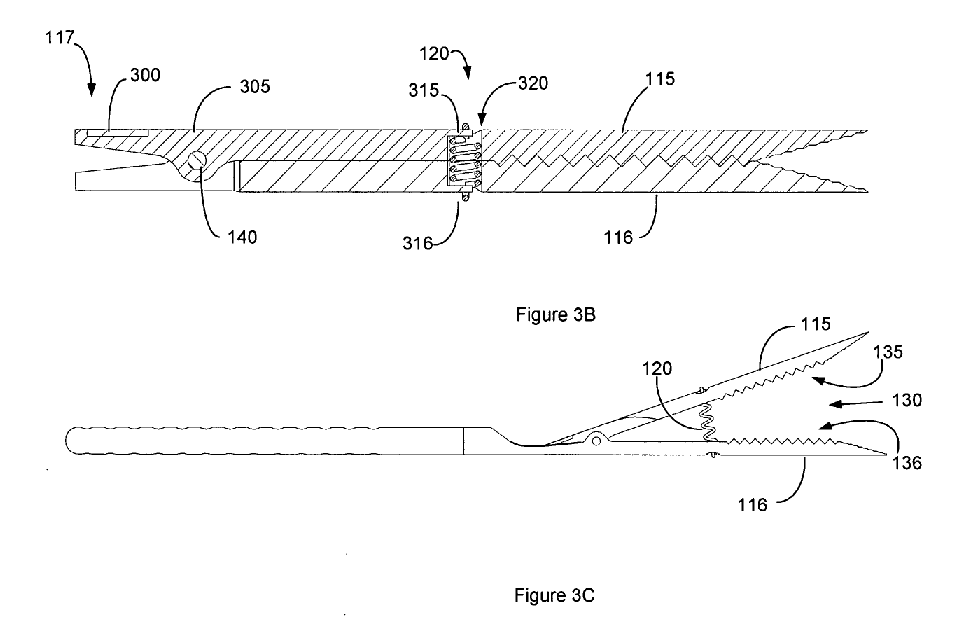

In the arrangement of FIG. 1, the force is applied to a first jaw to effect a movement of that first jaw away from the second jaw. The first jaw comprises a handle or lever 117 which on application of a force downwardly thereon in a direction toward the jaw 116 will effect a movement of the first jaw 115 away from the second jaw 116. As shown in FIG. 3B, the handle 117 may comprise a channel or notch 300 provided in an upper surface 305 thereof to facilitate engagement of an external force applicator with the handle, this notch may feature a soft insert or, as shown in FIGS. 2D and 2E, a knurled finish enabling better grip. The body portion 160 could be dimensioned in the region of the handle 117 to provide a channel 301 within which the handle may be located. In this way the depth of movement of the handle 117 may be increased to facilitate a larger opening of the jaws. The channel 301 could provide an aperture through which the handle may pass to increase further the opening angles of the two jaws to one another.

FIGS. 2D and 2E show a modification to the simple lever 117 heretofore described in FIG. 1. In this arrangement, an angled member 265 is used to effect an opening of the jaw. The angled member 265 is coupled at one end to the lever 117 and the other end is received within a channel 270 formed within the body 160. The channel has a length sufficient to allow the angled member to extend, on application of a force onto the angled member, rearwardly into the channel in a direction away from the mouth 130 of the grasper. The member 265 slides proximally when actuated and springs forward closing the jaw when released. In this way a separate spring may not be required to bias the jaws closed. The angled member 265 may be manufactured from a shape memory or plastic metal, polymer or composite thereof.

FIGS. 2F through 2H show a modification to the grasper described heretofore where a first 115a, 116a and second set 115b, 116b of jaws are provided at each end of the grasper. By providing a jaw of the same characteristics at either end of the grasper it is possible to provide a reversible tool. In another arrangement of a dual-headed grasper, the jaws at a first end are provided with different characteristics in that a first set could be sharpened and a second set provided with an atraumatic tip to allow flexibility in usage. Desirably at least one of the heads is provided with sharp jaw ends so that they can penetrate the abdominal wall or target tissue as required for fixation. Each of the heads may be provided with a fixed orientation relative to the other of the heads. In another configuration the heads may be moved relative to one another. This could be by means of rotation of a first head relative to a second head and/or a change in the angular orientation of one to another. In a first configuration the body may be provided of a flexible material so as to allow a flex of the body to change the angular orientation. In the exemplary arrangements illustrated each of the two heads are provided on a separate segment of the grasper body and a coupling member is provided therebetween to effect a coupling between the two segments. The coupling member may be rigid or flexible to enable the angle between the jaws to vary.

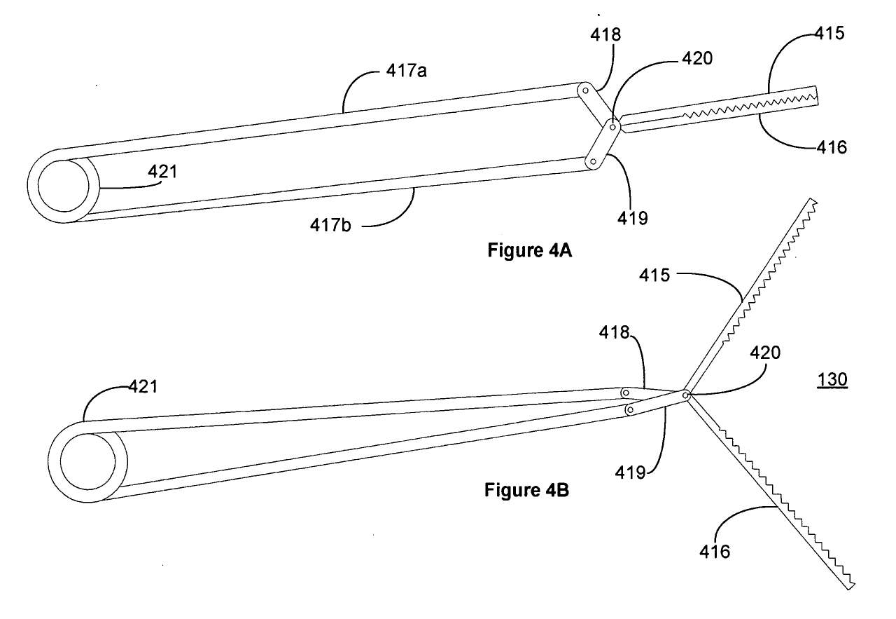

In another arrangement the force may be applied to each of the first and second jaws such that the separation of the jaws from one another is effected by a relative movement of each of the first and second jaws. Such an arrangement is shown in FIG. 4A where first 415 and second 416 jaws are cantilevered off a single pivot 420 which is individually coupled to first 417a and second 417b arms via first 418 and second 419 pivot couplings respectively. The arms are integrally formed about a loop 421 provided at an end of the device distally located from the mouth 130. The wind of the loop biases each of the arms coupled thereto to effect a normally closed position on the jaws—as shown in FIG. 4A. By compressing the two arms towards one another, the pivot couplings 418, 419 move towards one another to effect a scissors opening of the jaws to separate and define a mouth 130 within which an organ or tissue may be grasped or clamped. A grasper of such an embodiment may be sutured at the rear spring loop 421 or coupled at that rear loop 421 to a microtrocar or pin-type device.

The biasing of the jaws may be provided by a compression spring located between the pivot pin 140 and the mouth 130 of grasper head 110. Details of the location of the spring are shown in FIG. 3B wherein it is evident that the spring 120 may be coupled to each of the first 115 and second 116 jaws through for example a coupling feature 315, 316 provided on each of the first 115 and second 116 jaws. FIG. 3A shows a plan view of three different variations of mounting arrangements for the spring 120 within the jaws, 115 and 116. The spring is mounted within a spring housing 320 having an internal diameter substantially equivalent to the outside diameter of the spring. In a first arrangement the notch feature 315a includes an anvil arrangement at the end to prevent a slipping of the spring off the end of the notch. In a second arrangement the notch feature 315b comprises a circular stop member to again increase the diameter of the notch at the end of the notch to prevent spring slippage. In the third arrangement which is a simpler arrangement, the notch 315c is planar and slippage is prevented purely by the forces exerted downwardly by the spring.

By locating the compression spring to the second side of the pivot pin 140, that side proximal to the mouth 130, a smaller spring may be used to achieve the required biasing force. The force is related to the intended use of the grasper, specifically the nature of the organ/tissue which is to be grasped by the grasper. Desirably the force is sufficient to ensure that once located within the jaws, the biasing of the jaws towards one another will effect a retention of the organ/tissue therein without any slippage. At the same time, the force cannot be so great as to effect a cutting or damaging of the organ/tissue. It will be appreciated that other springs, such as for example a torsion spring, could be used in applications where smaller forces are required to those that are achievable using compression springs. Other configurations may comprise providing a spring about the pivot point, the spring extending in the same direction as the pivot so as to be substantially perpendicular to the longitudinal axis of the device. In such an arrangement the spring may comprise arms extending outwardly from the spring body towards the mouth of the device. By forming the jaws about these spring arms or coupling separately formed jaws to the spring arms, the spring will effect a biasing of each of the two jaws towards one another.

The jaws may comprise teeth 135, 136. As shown in FIG. 3C, desirably such teeth, if provided, are located in the mouth region of the jaws such that on presentation of the grasper to the organ, the teeth will engage with and effect a retention of the organ within the mouth of the grasper. In the arrangement of FIGS. 1 through 4, each of the first 115 and second 116 jaws have teeth 135, 136. As shown in the closed orientation of FIGS. 1 to 3, excluding FIG. 3C, the teeth of the first and second jaws are desirably configured such that on bringing the first and second jaws towards one another, the teeth of the first jaw will inter-engage with the teeth of the second jaw so as to allow a complete closing of the mouth of the grasper.

In another arrangement, one of the two jaws comprises teeth and the second of the two jaws provides a planar surface. In yet another arrangement, shown in FIG. 1B, a first jaw comprises first 136a and second 136b sets of teeth that extend alongside each other but are offset from one another enabling both jaws to be the same part which assists in a manufacturing process.

At least one of the jaws, and as shown in FIG. 2 both jaws, comprise a tapered lead-in portion 255, 256, tapering from an end portion 200 of the grasper head inwardly towards the mouth region 130. In this way, in a closed orientation, the jaws are separated from one another at the lead-in portion so as to provide maximum opening of the jaws. The lead-in portion may comprise a set of teeth 235, 236 of finer grade to that of the teeth located within the mouth portion of the grasper. The teeth may be provided in a traumatic or atraumatic configuration depending on the retraction application or the value of the organ/tissue being grasped by the grasper.

The length of the jaws may be the same or different. While the exemplary arrangements illustrate each of the first and second jaws being coincident at the end of the grasper, in modifications to that illustrated one or other of the two jaws could project beyond the other. Desirably in such a configuration, the lower jaw would have a length so as to project beyond the end of the upper jaw. This could advantageously assist a positioning of the lower jaw under the organ or tissue to be grasped. On suitable overlap between the lower jaw and the organ/tissue, the upper jaw could be clamped down onto the tissue so as to engage or grasp the organ/tissue between each of the first and second jaws.

The grasper further comprises a body portion 160 coupled or integrally formed with the head portion 110. The body portion desirably provides a coupling contact for effecting a securing of the grasper at a desired position within the abdominal cavity. The body portion could be provided as a separate moulded component to the head portion and coupled separately thereto. In another arrangement the body could be over-moulded onto the lower jaw 116. In a further arrangement the lower jaw 116 and body 110 can be moulded together as one piece.

A soft elastomeric rubber or plastic contact surface can used to aid the working grasper grip. These sections can also be roughened/sandblasted to achieve the same.

In a first arrangement the body portion extends longitudinally along the longitudinal axis A-A′ (shown in FIG. 2) away from the head 110 and has a length substantially greater than the length of the head. In such an arrangement, the body portion may comprise a plurality of coupling surfaces 161 spaced apart along the body portion and configured to cooperation with a separate coupling tool. In a first arrangement, shown in FIG. 2C, the coupling surfaces are provided as a plurality of individual coupling ports 162 extending through the body portion from an upper surface 163 to a lower surface 164. Each of the coupling ports desirably comprises a tapered mouth or chamfered entrance 165 to assist the presentation and location of the separate coupling tool within the coupling port. In such an arrangement, movement of the coupling tool from a first coupling port to a second coupling port requires a complete separation of the coupling tool from a first coupling port of the grasper and the re-presentation of the coupling tool to a second coupling port of the tool. However, as was detailed above, the coupling ports feature a radiused or chamfered entrance to enable easier coupling location and the body portion, or segments of it, could also be provided as a transparent structure to achieve the same ease of coupling. Side view location of the coupling ports is further enhanced with a round body section. The coupling ports may comprise an inner coating formed from a resilient material that will deform on presentation of the coupling tool to and through the port, but will then compress back on the tool once it has passed through the port to maintain the tool in location within the port. This arrangement may be advantageously employed with a coupling tool of varying diameter. Such an example could comprise a lead in portion of greater diameter than the remainder of the tool such that on presentation of the tool, the lead-in portion effects an expansion of the diameter of the coupling port through deforming the resilient material. The subsequent lesser diameter body of the tool does not effect the same force on the resilient material which will thus return to its normal shape, enveloping the tool therein.

In another arrangement the length and diameter of the coupling ports may be configured to restrict movement of an inserted coupling tool relative to the device until each are substantially perpendicular with one another. In this way, any deviation of the two from the normal will effect an interlock between edge portions of the coupling port and the tool, preventing a removal of the tool from the port.

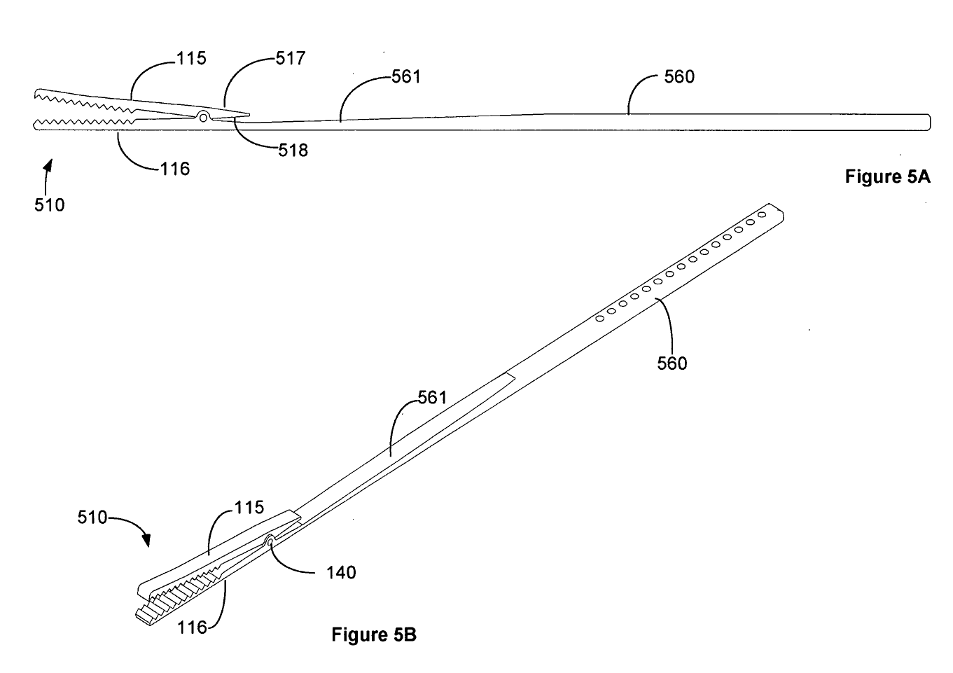

The arrangements of FIGS. 1 though 4 show the body have a rounded surface. In a modification to such an arrangement, shown in FIG. 5, the body portion may be provided with straight surfaces. The use of such a rectangular or square body portion 560 enables it to be sutured directly to the inner abdominal wall in an easier fashion and provides increased contact area between the body and tissue. The body sections of each embodiment may feature a stiffness gradient becoming more flexible at the end distal to the mouth 130. This may provide better coupling to the abdominal wall by adjusting to the desired shape. By providing the body portion in an elastomeric or otherwise flexible material that portion that contacts the abdominal wall may flex to adopt the contours of the abdominal wall. In this arrangement, the upper jaw 115 is still fixed through use of a pivot pin to the lower jaw 116. In this arrangement however, the orientation of the first and second jaws is optimised to enable the placement of a torsion spring inside the two jaws to maintain a normally closed position. The mandrel on which the torsion spring is positioned also serves to couple the top and bottom jaw.

In the arrangement of FIG. 5 an upper surface 561 of the body portion tapers downwardly in the vicinity of the head portion 510 of the grasper. In this way a lower surface 518 of the handle 517 has a greater distance to travel before abutting against this upper surface which allows for greater movement of the jaws 115, 116 apart and hence allows for the grasper to be used with larger pieces of organ/tissue.

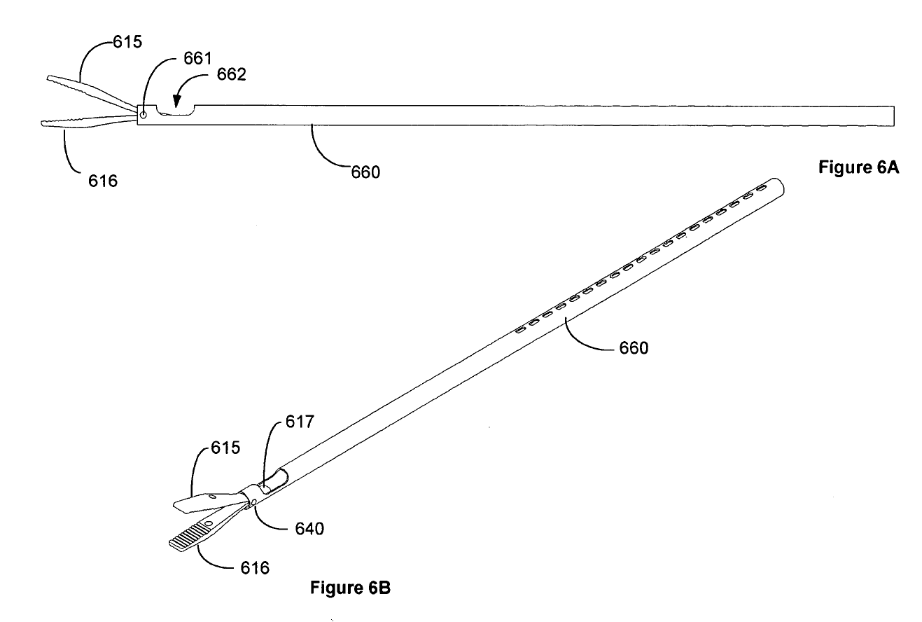

In the arrangements of FIGS. 1 through 5, excluding FIGS. 1B to 1I the body portion and the lower jaw may be formed as an integral element. The upper jaw was fixed to this integral element via a pivot pin and could therefore move relative to the lower jaw to facilitate an opening and closing of the mouth of the grasper. The construction of the grasper therefore required three elements: the integral body/lower jaw element, the upper jaw element and the pivot pin. FIG. 6 shows an alternative construction where the body portion 660 is formed separately to each of the first and second jaws. In this arrangement the body portion is fabricated from a single tubular element which is provided with an aperture 661 for receipt of the pivot pin 640 and a cut-out 662 in the vicinity of the pivot pin aperture 661. On mounting each of the first and second jaws 615, 616 to the body portion 660 using the pivot pin 640, a handle portion 617 of the upper jaw is accessible within the cut-out 662. The application of a force on that handle portion 617 effects a movement of the handle portion downwardly into the interior portion of the body 660, with a corresponding movement of the two jaws apart. Similarly to the previously described arrangements, each of the two jaws are biased towards one another such that they will normally adopt a closed position. The jaw profile is optimised for maximum strength and opening. A compression spring can be mounted inside the jaws to maintain closure or a torsion spring can be incorporated under the opening effectors, or both methods can be combined.

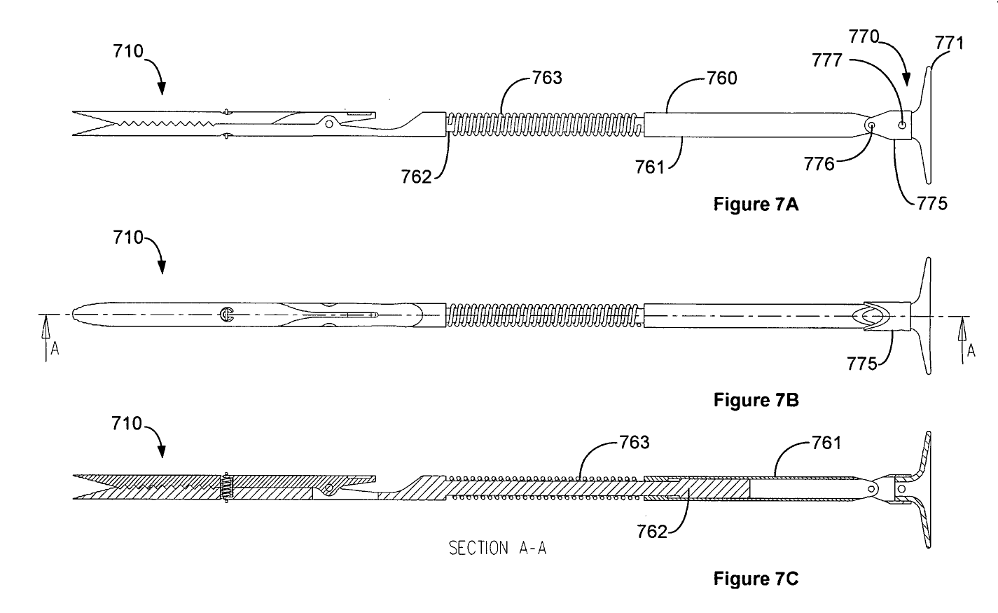

As shown in FIGS. 7 to 9, the body may be configured to be moveable relative to the head of the grasper. In a first arrangement, shown in FIG. 7, the body 760 may be moveable along the longitudinal axis of the grasper in a direction parallel to the line A-A shown in FIG. 7B. In this exemplary arrangement the body portion is provided in a multi-element construction comprising a telescoping arrangement whereby an outer sleeve 761 is moveable along an inner sleeve 762. The movement is controlled by a spring 763 arranged about the inner sleeve. Such an arrangement may allow for a compression of the body towards the head so as to effect a reduction in length of the grasper. Such an arrangement is advantageous in circumstances where the grasper is to be secured against a surface. By spring loading the body portion relative to the head, a movement of the body towards the head can be used to reduce the length of the grasper. This allows a location of the grasper within the abdominal cavity. The release of the biasing force that effects a compression of the body will allow the grasper to attempt to return to its normal elongated state. If the available space is not sufficient the grasper will be retained in position by being positively biased against the surface preventing the extension to its normal position. Such an arrangement is advantageously employed in circumstances where the grasper is to be used as a “strut” type support to leverage a particular organ into a desired orientation.

The body portion 760 may be coupled to a surface engagement portion 770 which provides a contact surface for engaging against the abdominal cavity surface. In the arrangement of FIG. 7, the surface engagement portion 770 comprises a flexible portion 771 which may be contracted to reduce its dimensions but which on deployment within the abdominal cavity may be extended to provide a substantially enlarged contact surface for contacting against the abdominal cavity surface. In the exemplary arrangement this enlarged contact surface is in the form of a disc having a major surface that is substantially perpendicular to the longitudinal axis A-A of the grasper. It will be appreciated however that the specifics of the arrangement shown are not intended to be limiting in any fashion. By providing this flexible surface or membrane, the surface-engaging portion can be contracted during an introduction of the grasper through a trocar into the abdominal cavity but on full insertion thereof, can be extended to facilitate generation of a larger surface for distributed weight loading. While not necessary in all uses, it is possible to provide the flexible portion as an inflatable or otherwise temporarily rigid element and such an arrangement should be considered as part of the present teaching.

The surface engagement portion 770 may be moveable relative to the head portion 710 of the grasper such that it may be angularly orientated relative to the head portion. This may be achieved by having the surface engagement portion moveable to both the body portion and the head portion as shown in FIG. 7 or simply having the surface engagement portion moveable relative to the head portion. In a first arrangement, shown in FIG. 7, the surface engagement portion 770 is moveable in two axes and has two degrees of freedom relative to the body portion 760. This is facilitated in this exemplary arrangement by use of a coupler 775 that is independently coupled to each of the body and the surface engagement portion. By providing a first pivot coupling 776 to the body, it is moveable relative to the body. The outer sleeve 761 can also rotate relative to the inner sleeve 762.

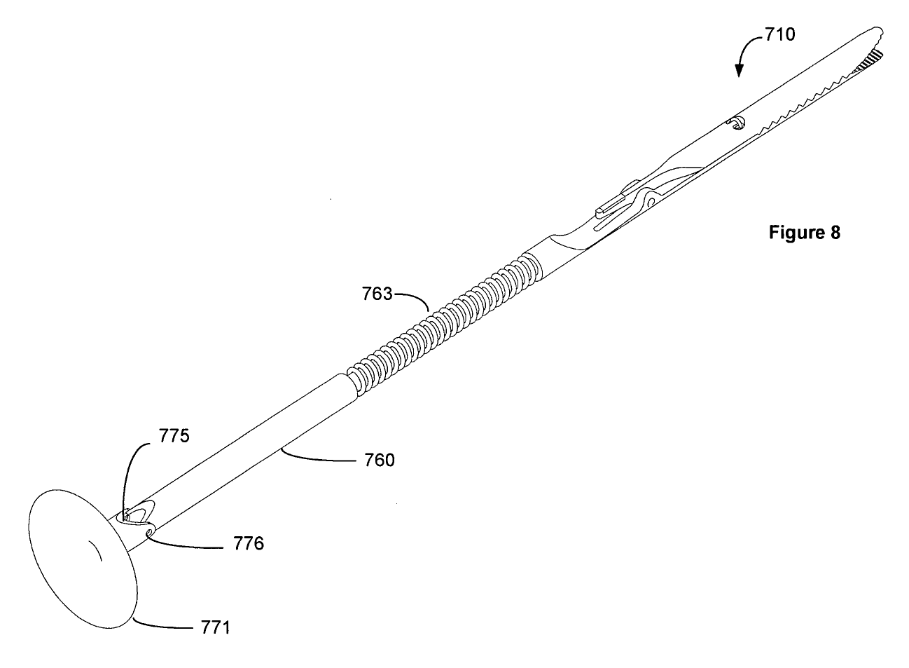

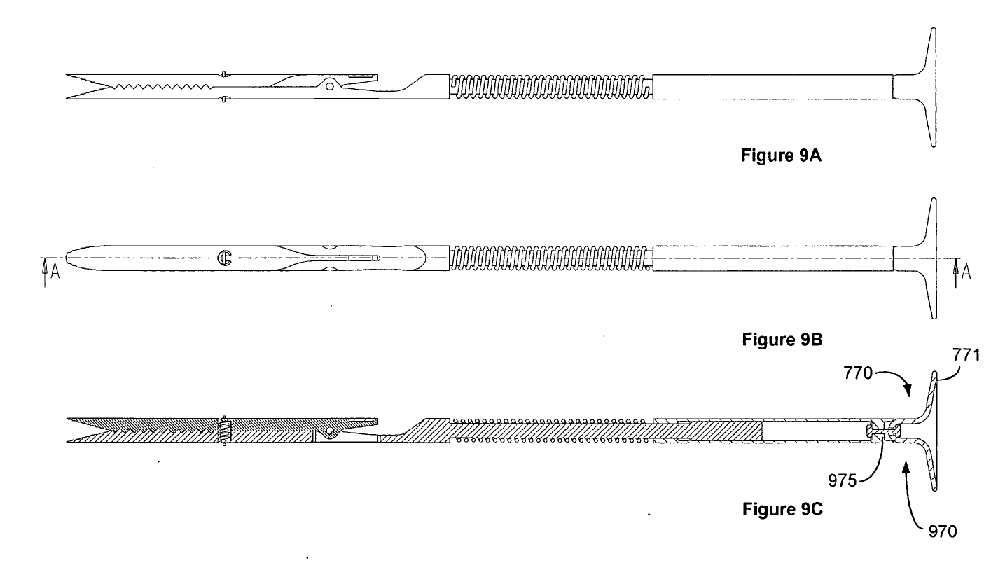

FIG. 9 shows a modification to that of FIGS. 7 and 8 wherein the surface engagement portion may be rotated relative to the head portion. Similarly to that previously described a sprung loaded proximal section or body portion is provided with a universal proximal support. The support or surface engagement portion 770 comprises a deformable membrane 771 as per the previous embodiment but can be rotated and held in place by a dog bone shaped connector 975. A universal or constant velocity type joint may also be used to achieve the same effect.

In the arrangements described heretofore, the surface engagement portion may be moved so as to be offset from the longitudinal axis of the grasper or may be configured to be both rotatable and angularly offset from the longitudinal axis of the grasper.

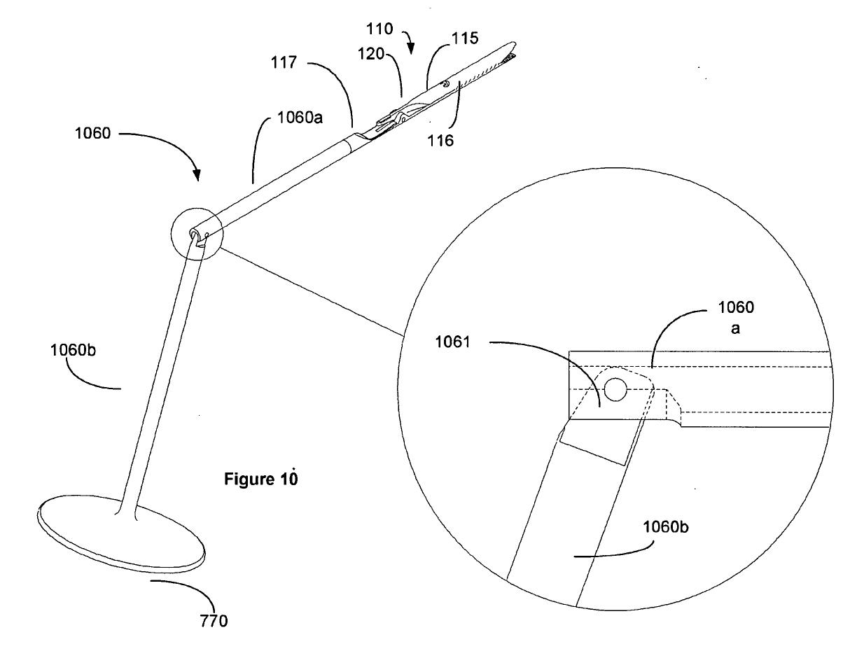

FIG. 10 shows an alternative arrangement for changing the orientation of the surface engagement portion relative to the head of the grasper. In this arrangement the body portion 1060 may be segmented to comprise a head proximal body portion 1060a and a head distal body portion 1060b. The surface engagement portion 770 is coupled to the head distal body portion 1060b. Such coupling may be a fixed coupling or could also allow movement of the surface engagement portion relative to the head distal body portion 1060b. The first and second segments of the body portion may be moveable relative to one another, such as for example by using a pivot coupling 1061. In this way a change in orientation of the first segment relative to the second segment may be effected. By using the exemplary pivot coupling the degree of freedom of movement is restricted, but as will be appreciated by those of skill in the art other coupling mechanisms between each of the segmented portions could be used if more relative movement was required.

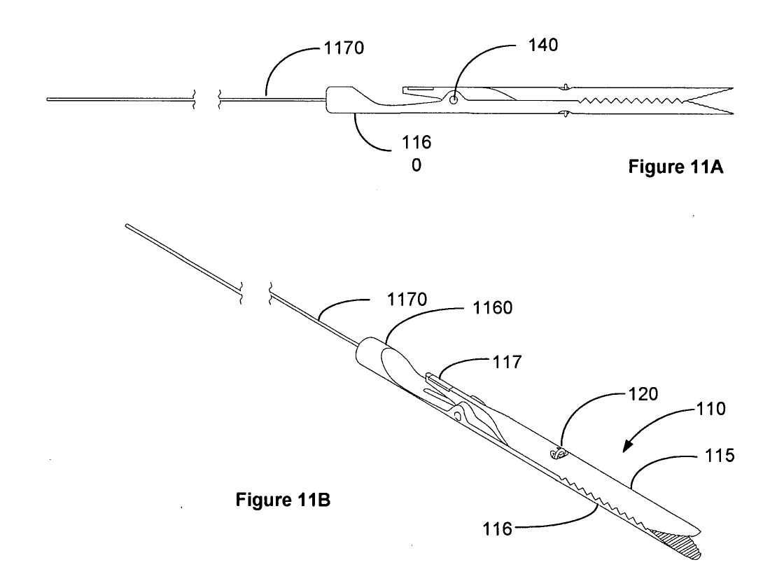

In another arrangement, shown in FIG. 11, the body portion 1160 is coupled to a suture 1170 which operably may be used to tether the grasper so as to secure a retained organ/tissue at a suitable location. In this arrangement the suture provides a flexible tether between the head portion 110 and the structure to which it is being retained against. Use of such an arrangement typically requires a passing of the suture through the abdominal wall, or via a provided cannula. As the length of the suture can be modified depending on the distances between the organ being grasped and the tether to which it is being secured against, the length of the body portion 1160 is not as long as it was in other previously described arrangements.

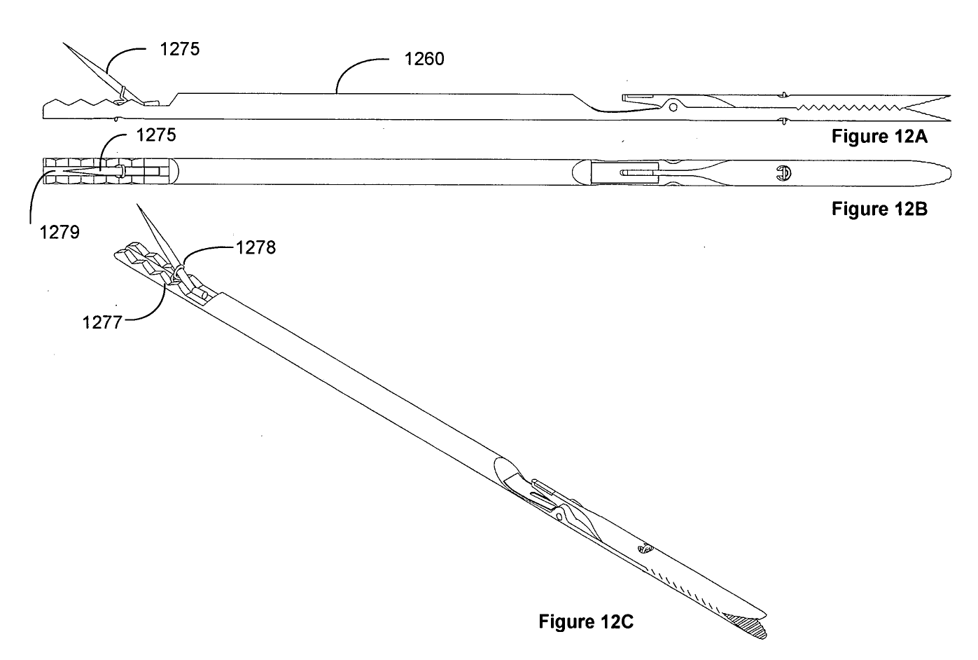

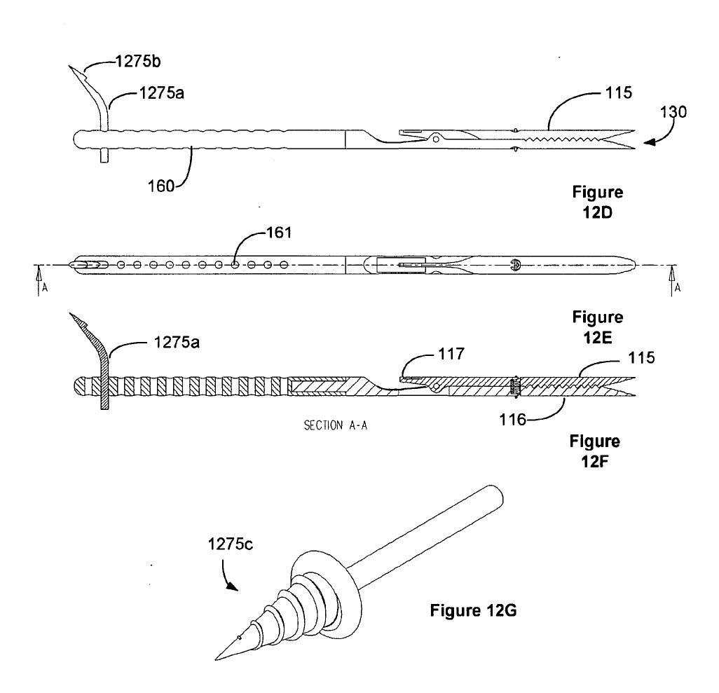

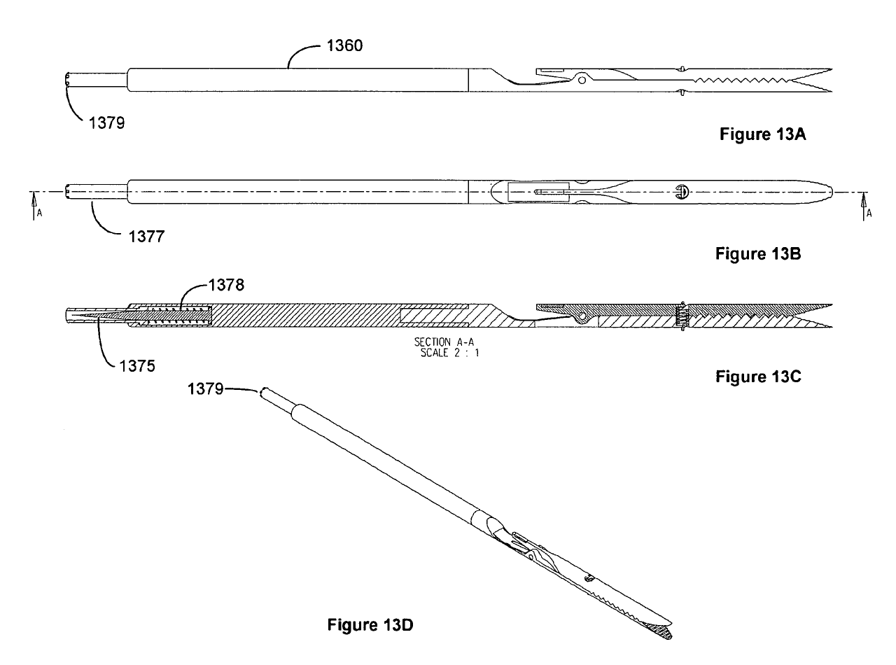

In a further arrangement, examples of which are described with reference to FIGS. 12 and 13, the body portion 1260, 1360 comprises a deployable needle 1275, 1375 so as to allow a fixing of the grasper to an inner abdominal wall or other desired feature.

In a first arrangement shown with reference to FIGS. 12A through C, the needle 1275 is mounted relative to the body portion 1260 so as to be pivotable from a first position wherein the needle is located within the extremities of the body portion to a second position wherein the needle is moveable away from the body portion and is angularly positioned relative to the body portion. The needle may be barbed to provide better attachment if required. On adoption of the second position the needle may be presented to the inner abdominal wall or other feature within the cavity to effect the necessary securing of the grasper. The grasper can thereby, using an embedding of the needle to the wall or other feature, be fixed to the inner abdominal wall or other desired feature. The needle is sprung against a jaw element 1277, using a spring 1278 coupled between the needle 1275 and the jaw element, to secure the tissue. The jaw element 1277 defines a channel 1279 within which the needle may rest during a non-deployed state. The dimensions of the channel 1279 are desirably greater than the outer diameter and length of the needle such that the needle, when located within the channel, does not project beyond the channel where it may cause inadvertent damage. The needle of this arrangement is desirably provided with a bend such that it can pivot about the bend to be moved to the deployed state. In an alternative configuration, not shown, a spring-loaded claw replaces the needle. The claw is in the closed position by default and may be actuated in a similar manner to the front jaws. The nose section in front of the claw may be fabricated from a flexible material so that when pressed against the abdominal wall the claw section is revealed and moves back when further force is applied in the direction of the abdomen. This then serves to fix the device with the load being transferred from the retracted organ/tissue further embedding the claw. The claw may also be spring loaded so that when the actuator is depressed the claw is revealed and anchoring achieved. The sharp edge of the claw is hidden when not in use to prevent inadvertent damage or grasping of adjacent tissue while positioning the distal end.

In an alternative configuration shown in FIGS. 12D through E, a needle 1275a is formed separate to the body 160 of the grasper and is mounted within an appropriate coupling port 161 at the time of use. In this arrangement, the separate needle is provided with a barb 1275b, and is designed with a predefined or adjustable angle so that it can fit through a 5 mm trocar. The barbed section 1275b is fixed into the abdominal wall and can then be coupled to the grasper. A screw-type coupler 1275c is also shown in FIG. 12G as an example of an alternative to the barbed needle 1275a.

In yet a further modification, albeit not shown, the needle is provided beneath a moveable sleeve that is configured to move along the body of the grasper so as to reveal the needle. By providing the needle in a shape memory alloy where it will revert to its original shape on removal of a bias force (as would be provided by the cover of the sleeve) or by having the needle positively biased by a spring, the movement of the sleeve will effect a deployment of the needle to an actuated position whereby it may be inserted into the tissue or abdomen wall as appropriate. The sleeve may be provided in a spring loaded arrangement whereby it will normally tend to revert to the shielded position covering the needle. Actuation of the movement of the shield may be effected by providing a force from another device. To facilitate inter-engagement of the two, the shield may be provided with a notch or receiving member for coupling to the external device.

In the arrangement of FIG. 13, an alternative housing for the needle is shown. In this arrangement the needle 1375 is substantially co-linear and is normally sheathed or otherwise protected to ensure inadvertent contact of the needle with organs/tissue within the abdominal cavity is not achieved. Such a sheathing may be provided by locating the needle 1375 within a spring loaded needle protector 1377. Such a protector may include one or more grips 1379 provided at an end surface thereof to prevent it slipping relative to the abdominal wall. On application of a load to the protector, the protector is configured to retract against a spring 1378 provided within the body portion 1360 so as to be received itself within the body portion 1360. On movement of the protector into the body, the needle is thereby exposed and can be used in a puncturing of tissue and an anchoring of the grasper.

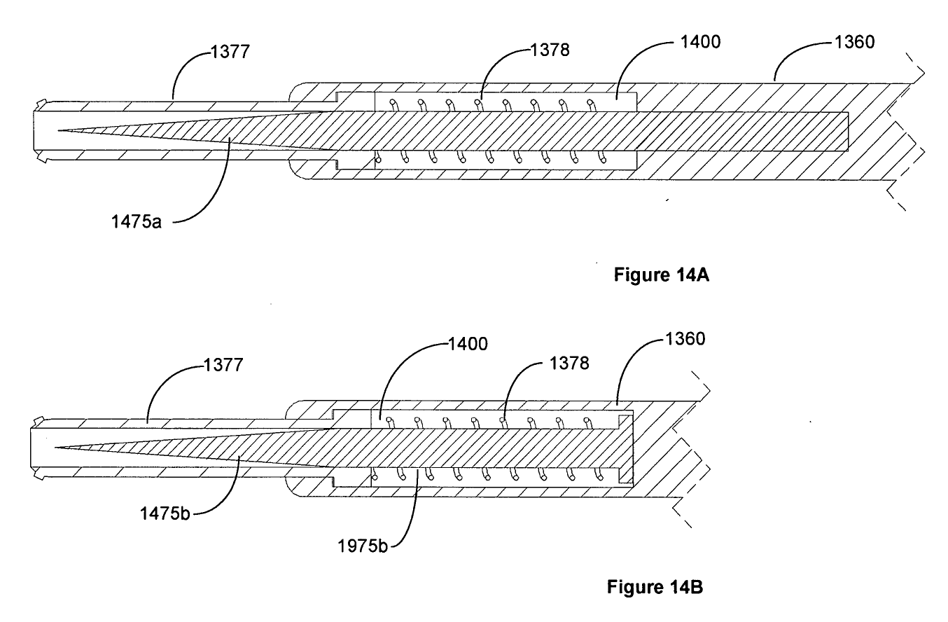

FIGS. 14A and 14B show alternative but non-limiting means of securing the needle to the main body of the grasper. In FIG. 14A the needle 1475a extends through an inner cavity 1400 provided within the body 1360 and is secured within the body proper using a fixing adhesive or the like. The inner cavity serves in use to receive the protector and guides the protector on application of a force against the protector into the body 1360. In FIG. 14B the needle 1475b is secured within the cavity itself as opposed to extending further through and into the body as in FIG. 14A.

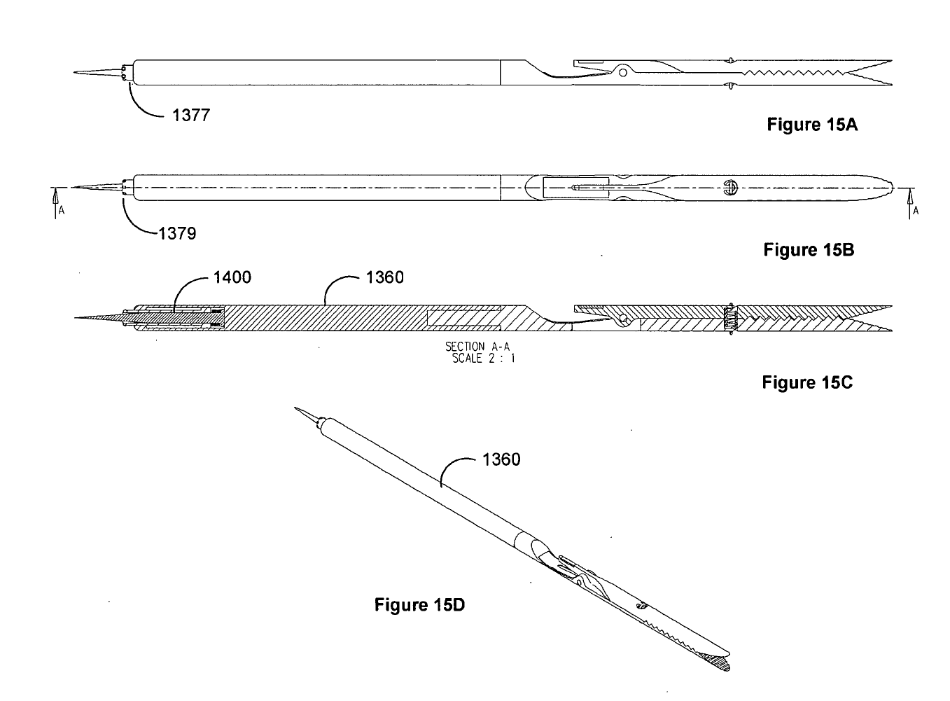

FIG. 15 shows how the needle arrangement of FIGS. 13 and 14 will appear in a deployed arrangement. As is seen the length of the cavity 1400 is less than the length of the protector such that when the protector 1377 is retracted into the cavity 1400, it is not fully received and still projects somewhat beyond the end of the body portion 1360. In this way the needle may be inserted into the tissue up to a depth commensurate with the location of the grips 1379 which will then prevent slippage of a received grasper against the tissue to which it is embedded.

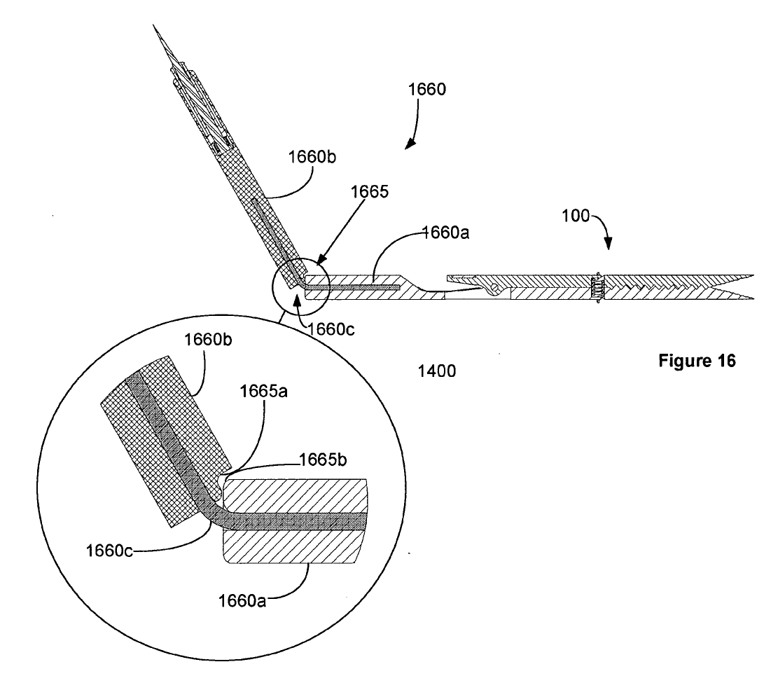

FIG. 16 shows an example of how the surface engaging portion embodied by the needle arrangements of FIGS. 13 to 15 may be moved relative to the head portion 110 of the grasper. In this arrangement, the body portion 1660 is segmented in a similar fashion to that described with reference to FIG. 10 to comprise a head proximal body portion 1660a and a head distal body portion 1660b. The two are interconnected using a shape memory actuator 1660c a portion of which is coupled to each of the proximal and distal body portions. The grasper may be initially provided in a configuration where each of the proximal and distal body portions are co-axial so as to allow for the grasper to be passed though a trocar. On complete insertion of the grasper into the abdominal cavity, the grasper may then have its orientation changed to allow adoption of the bent configuration, shown in FIG. 16, to enable oblique fixation of the grasper. By providing a shoulder 1665 or other locking mechanism, the distal body portion 1660b cannot be orientated to a position where it achieves a perpendicular relationship with the proximal body portion 1660a.

Heretofore the movement of the two jaws relative to one another has been described with reference to application of an external biasing force onto a handle 117 of at least one of the two jaws to effect a pivoting of the jaws relative to one another. In another configuration one or both of the jaws could be configured to incorporate an integral biasing force. This could be achieved for example by forming one or both of the upper or lower jaws from a torsion spring which would serve to bias the jaws towards one another. This could be adapted so that the top and bottom jaws are both made from a spring.

FIGS. 1B through 1I show yet another alternative mechanism. In this arrangement the grasper may be advantageously employed in grasping the gallbladder and can be wholly inserted in through a trocar for a laparoscopic procedure. Similarly to FIG. 1A each of the first 115 and second jaws 116 are biased relative to one another through use of a spring 120 to adopt a normally closed position. The lower and upper jaws are coupled to one another and each are moveable relative to the other to adopt the open position. In this arrangement a linkage between an actuator 191 and each of the lower 116 and upper 115 jaws are provided. The linkage comprises a first set of actuating members 192 pivotably coupled to a second set of actuating members 193 in an opposing scissors-linkage arrangement. The second set of actuating members 193a, 193b may be individually coupled to each of the upper jaw 115 and lower jaw 116 or an integral part thereof. Each of the first set of actuating members 192a, 192b are coupled to the actuator 191.

The actuator or push wire 191 extends back to the body portion 160. The actuator 191 itself may be formed from a shape memory material to achieve the desired default closure which eliminates the requirement for a spring. A channel 301 is defined in an upper surface of the body portion to provide access to the push wire. The wire is desirably formed from a shape alloy such as Nitinol and has a set shape but may also be comprised from a non super-elastic material. The actuator is moveable in a direction substantially transverse to the direction of opening of the jaw. In this arrangement it may be considered as being moveable in a direction substantially parallel with the longitudinal axis of the device. In use, a conventional grasper is then passed through the trocar and engages the shape set wire 191. The sleeve 194 around the wire constricts it into moving forward, thus pushing the jaw opening mechanism open. A spring 120 embedded into the grasper jaw forces the jaw closed once the shape set wire 191 is disengaged, or as previously described the shape set wire may force closure of the jaws. As shown in the variations of FIGS. 1B through 1F, variations in the shape of the actuator 190 may be employed depending on for example the specifics of the distance to be travelled, extent of opening of the jaws required and other similar parameters. The actuator may comprise one or more kinks provided in the surface. As shown in FIGS. 1G through 1I, as the actuator is fixed at one end 195 to the body such that compression of the actuator downwardly into the body only allows for its movement in a direction towards the mouth 130 of the device.

Each of the first set of actuating members are pivotably coupled to one another and are offset from one another such that action of the actuator thereon in a direction towards the mouth of the device effects a movement of the actuating members 192 towards the mouth. As each of these are also pivotably coupled to the second set of actuating members 193 this movement is translated into a corresponding movement of the second set of actuating members to effect an opening of the two jaws relative to one another.

In another arrangement, not shown, actuation is effected by a cam mechanism comprising first and second cam surfaces. The second cam surface may be actuated by movement of the first cam surface to effect a pivoting of the upper jaw relative to the lower jaw. Movement of the first cam surface is desirably effected or induced by action of the actuator 191 thereon, this movement being translated onto the cam surfaces driving the jaws open to extend the mouth 130. It will be appreciated that the illustrative views are not intended to limit the variations on the shape of the wire used to advance the jaw opening mechanism. While it is typical that actuation of the jaws would require action of an external force onto the wire 191, a certain configuration albeit not shown here, may include a slideable collar that could slide longitudinally along the body of the device between positions where it overlapped and thereby clamped the wire 191 so as to effect an opening of the jaws to a position where it did not overlap and the wire would revert to its normal non-biased state.

FIGS. 1J through 1L show another configuration where an atraumatic upper jaw 115b is coupled with a serrated lower jaw 116 which can be used to puncture tissue that is too large for the jaw to envelop completely within the jaw. It will be appreciated that in such a configuration the length of the lower jaw 116 is greater than that of the upper jaw to allow either for puncturing of the tissue by the lower jaw or to allow for the lower jaw to be orientated below an organ and extend under that organ to pivot it back for subsequent retention on a clasping down by the upper jaw 115b onto that lower jaw 116.

In this schematic, a polymer sleeve 195 is also shown as being incorporated to cover the actuating handle 117. It will be appreciated that the jaw configuration could be used with or without the sleeve. The location of actuation 196 is marked on the outer diameter of the sleeve 195. The sleeve 195 will be made of a relatively soft material to enable better grip of the actuation handle. While not shown in this configuration where a spring 120 is evident, in other arrangements the sleeve may also be positioned further along the jaw and replace the compression springs function for jaw closure. In such an arrangement, the wall thickness and material durometer is optimised to achieve the adequate closing force.

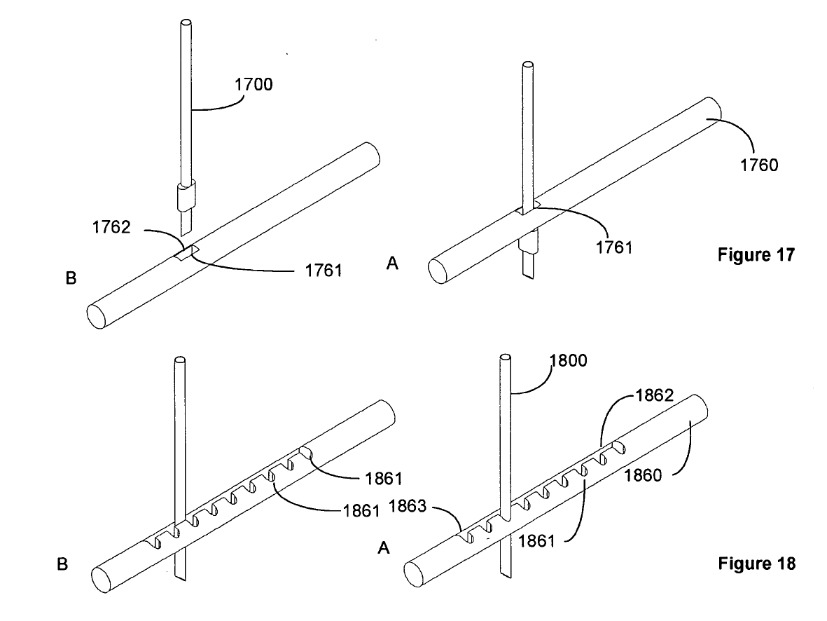

In the arrangements described heretofore coupling surfaces 161 spaced apart along the body portion and configured to cooperation with a separate coupling tool have been described with reference to a plurality of individual coupling ports 162. In an alternative arrangement shown in FIG. 17, the body portion 1760 comprises only one coupling surface 1761 which is provided similarly to that described previously is configured for co-operation with a separate coupling tool 1700. Such a latter arrangement is typically used where the intended ultimate location of the grasper within the abdominal cavity is well defined and the surgeon does not require the same level of flexibility as is provided by having a plurality of coupling surfaces. To achieve co-operation between the grasper and the tool, the tool is inserted into a slot 1762 defining a coupling surface. Rotation of the tool relative to the slot locks the tool within the slot preventing accidental separation of the two. This feature could also be applied to a plurality of slots. FIG. 18 shows a further modification comprising a plurality of inter-connected slots 1861 provided along the length of the body 1860, each of the slots providing a coupling surface 1862 for cooperation with the separate coupling tool 1800. In such an arrangement a change in the orientation of the coupling tool 1800 allows it to move from a first orientation to a second orientation. This change in orientation allows for the coupling tool to be slid along a channel 1863 within the body portion and moved from one slot to another. The tool 1800 may have a splined section which engages with the body through interaction with the keyhole shaped slots 1861. Rotation of the tool allows it to pass into the channel 1863 and passage to another slot. Each of the singular coupling ports may also be repeated along the shaft for applications where more flexibility is required.

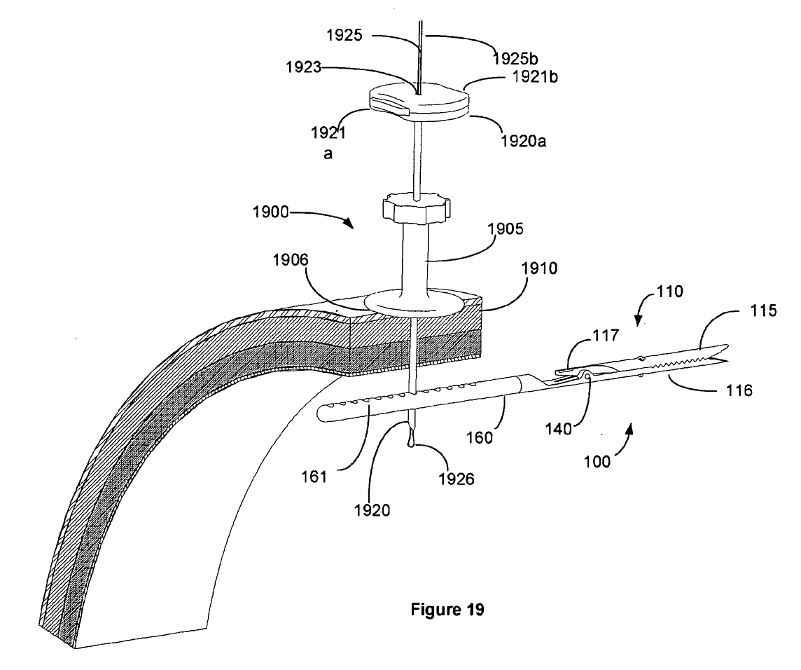

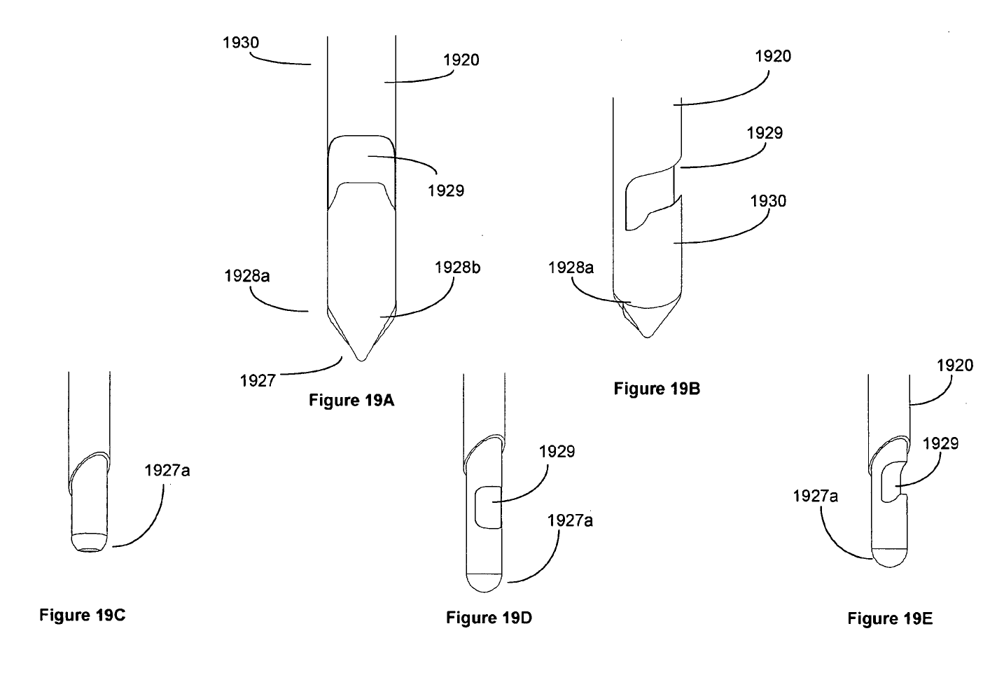

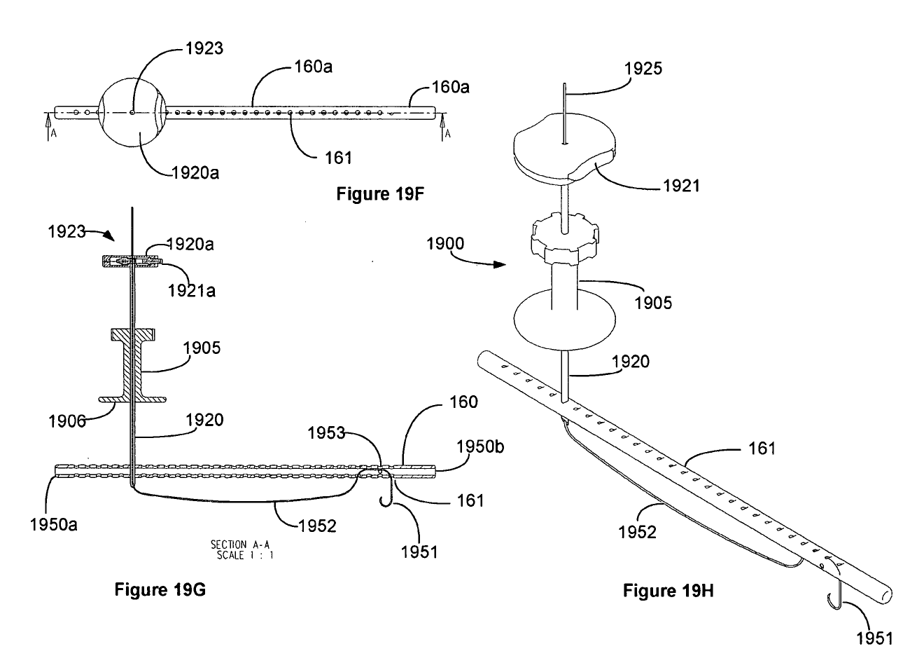

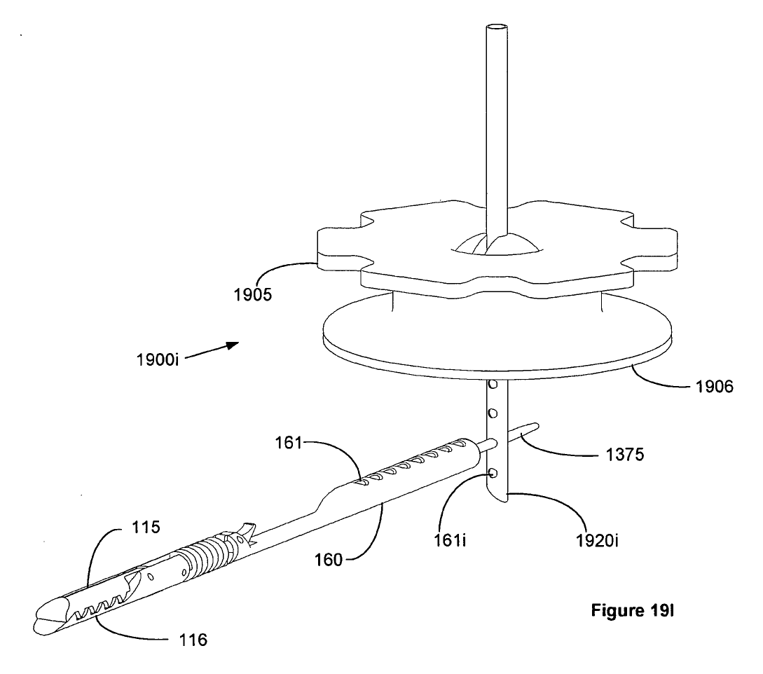

FIG. 19 shows how the grasper of FIG. 1A could be used. In this arrangement a microtrocar 1900 is provided which is stabilised on the abdomen 1910 via a movable anchor 1905. The bottom surface 1906 of the anchor may incorporate an adhesive for better anchoring or incorporate a textured surface to achieve the same. The microtrocar comprises a needle section 1920 which in use penetrates through the abdomen wall 1910 and engages with a slot 161 provided on the body 160 of the grasper. While not shown in this figure, such inter-engagement would typically be effected after the grasper head 110 had been used to grasp and retain a portion of a target tissue or organ. The subsequent movement of the grasper would effect a corresponding movement of the organ away from a surgical site. The organ would have a tendency to move back to that site and a force would be acting on the grasper to effect that movement back. This force is countered by the relative securing of the grasper through its inter-engagement with the microtrocar. The balance achieved between the two forces ensures that the grasped organ will retain its position within the abdominal cavity until it is released from contact with the microtrocar 1900.

This exemplary arrangement of a microtrocar that may be usefully employed within the present context comprises a sprung loaded suture grabber 1920a which has a handle that is biased inwardly onto a channel defined within the suture grabber. A suture catcher is looped 1926 and passed inwardly though the suture grabber such that first and second strands 1925, 1925b are provided within the channel 1923 onto which the handle is acting. By having the handle biased inwardly they will compress against the suture grabber strands 1925, 1925b preventing movement of them. Release of the handle allows the suture catcher to be moved within the suture grabber. The loop 1926 that projects through the end of the needle can be used to grab sutures within the body cavity, such as those described with reference to FIG. 11, and on grabbing the sutures can be used to effect a pulling of those sutures out of the abdominal cavity for subsequent manipulation. The sutures can then be fixed in place within the suture grabber by releasing the handle, and the suture tension adjusted. Other mechanisms for restricting the movement of the suture within the channel include the provision of a roller mechanism provided on Outer surface of the microtrocar which through a rolling can adjust the compression force exerted onto the suture within the channel. This could be provided by a cam or other arcuate surface disposed within the channel. Movement of the roller mechanism effects corresponding variance of the diameter within the channel through which the suture may pass. It will be appreciated that the less freedom that the suture has, the less movement that it has. In this way, the suture could still be moveable under application of a force such as for example through use of a puppeteer, but in the absence of that force it would retain its location within the channel.

Another simplified mechanism for securing the suture that has been extended out from the abdominal cavity would include the use of a cleat or notch feature against which the suture could be retained to prevent movement of the suture.

FIGS. 19A and 19B show details of various configurations of the end portion of the microtrocar needle section 1920. An atraumatic tip 1927 has a set of blades 1928a, 1928b extending outwardly from a central axis A-A′ and are provided to assist with insertion of the tip 1927 through the abdominal wall. A side port 1929 is provided in a side wall 1930 of the needle section 1920 and provides access to an interior volume of the substantially hollow needle. In use, this portion of the needle section will be disposed within the abdominal cavity and using this port 1929, it is possible to pass sutures externally of the body. FIGS. 19C through 19E show a modification to the tip configuration of FIGS. 19A and 19B where the tip is provided in the form of a bull-nose or blunt end portion 1927a as opposed to the sharpened end portion of the arrangement of FIGS. 19A and 19B. 1927a also features an opening through the blunt tip to allow a suture catcher to pass through.

FIGS. 19F through 19H show exemplary arrangements of use of the microtrocar 1900 used with a coupling feature 160A. The coupling feature 160a is similar to the main body portion of the grasper heretofore described in that it comprises a plurality of coupling ports 161 which may be used in combination with the needle end 1920 to inter-engage with the microtrocar. The coupling feature 160a comprises atraumatic ends 1950a, 1950b which could couple feature stabilizing balloons at either end. For gall bladder retraction the distal end may be positioned above the right lobe of the liver and a needle 1951 with suture 1952 attached is ran over a pulley pin 1953, through a coupling port 161 and back up through the microtrocar 1900 via the suture grabber 1920a. The gall bladder is then pulled to position and held in place via the sprung loaded microtrocar handle 1921a.

FIG. 19I shows a further configuration that may be advantageously employed in accordance with the present teaching. In this arrangement a microtrocar 1900i is provided with a needle-end 1920i having a plurality of apertures or coupling ports 1611 provided along its length. These individual coupling ports are dimensioned for the selective receipt of a distal end of a deployable needle 1375 therethrough. In this way it will be appreciated that the coupling of the grasper to the microtrocar is the opposite to that of FIG. 19 whereby, the grasper is configured to penetrate through the microtrocar as opposed to the microtrocar penetrating through coupling ports provided on the grasper. It will be appreciated that the grasper of this configuration also includes coupling ports 161 provided along its body 160 to allow the surgeon choice in the preferred coupling arrangement. In this way it will be appreciated that different features or elements described with reference to individual figures may be used with the graspers of other figures.

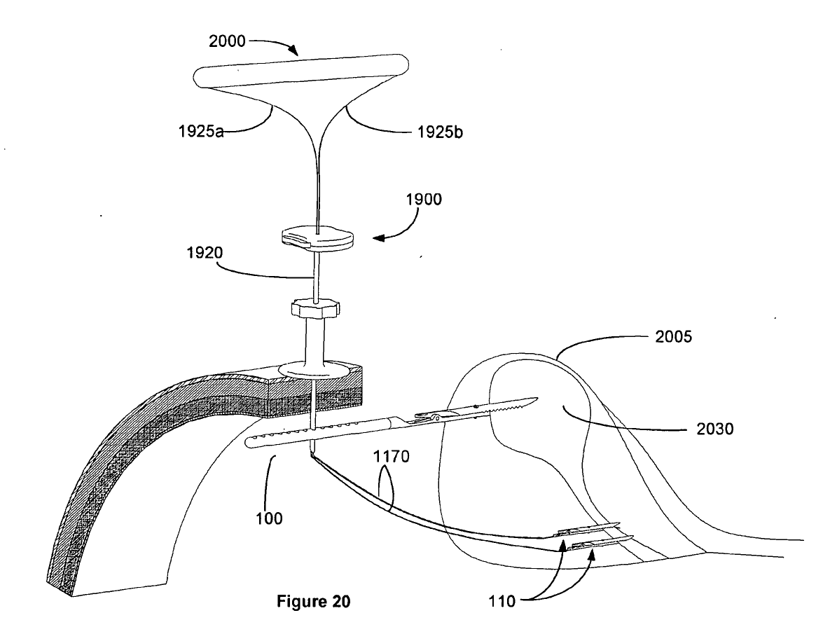

FIG. 20 is a schematic showing deployment of a plurality of graspers within an abdominal cavity. A drop in grasper 100 such as described in FIG. 1 is inserted into the cavity and used to engage the bulk or fundus of the gallbladder 2030 and the attached right liver lobe 2005. The microtrocar 1900 of FIG. 19 is used to secure the drop-in grasper. Two additional graspers 110, each of the type shown in FIG. 11 with suture control 1170 are provided to enable control on the fine movement at the root of the gallbladder and is attached externally to a ‘puppeteer” 2000. The heads of each grasper grasp the target area on the root of the bladder and can be manipulated through movement of the puppeteer.

The grasper may also be coupled to a solid shaft version of the microtrocar which does not utilise the suture grabbing capability but does feature an anchor similar to 1906 which can be similarly moved relative to the shaft and locked to maintain its relative position to the abdomen.

A further modification to a microtrocar that could be usefully employed within the context of the present teaching is a device comprising a needle portion and a contact surface that would operably engage against the body. Application of a force downwardly—through inter-engagement of the needle portion with a grasper is countered by the action of the anchor on the body surface. These two forces counter one another to ensure that a coupled grasper is retained in position.

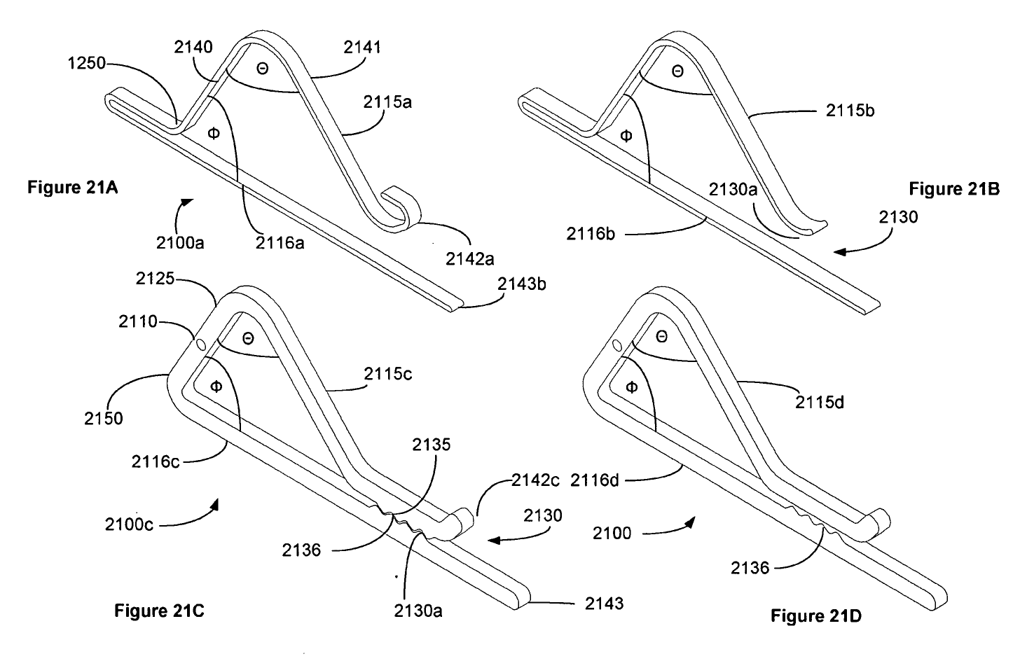

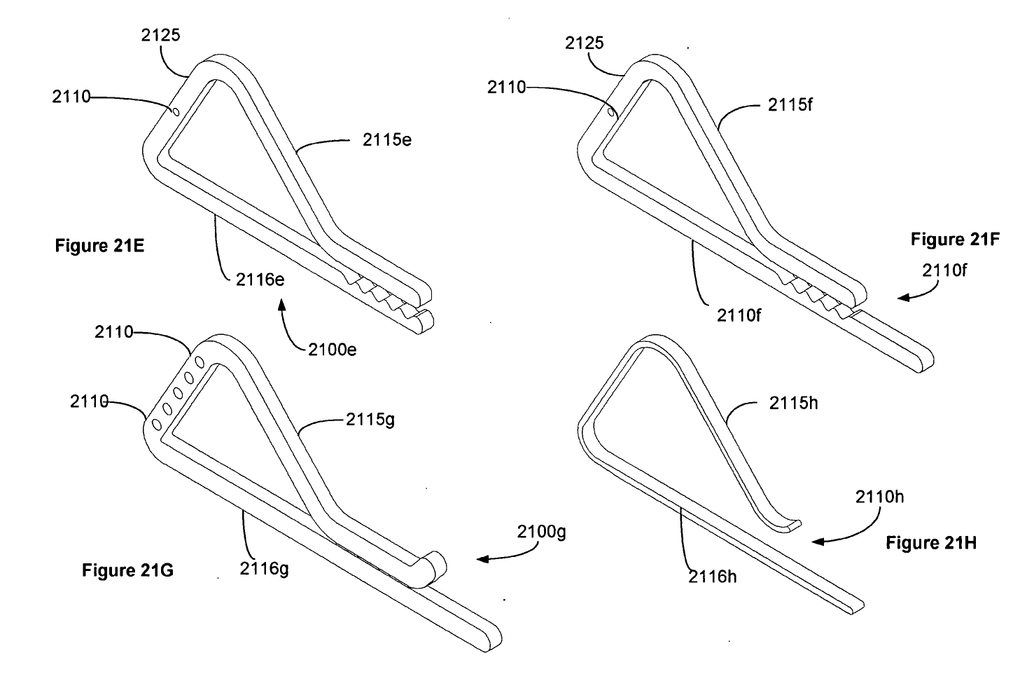

FIGS. 21A through H show further configurations of a surgical device in accordance with the present teaching which defines a clasping element 2100 again comprising a first 2115 and a second 2116 jaw that are biased towards one another. In these arrangements the jaws are integral with one another and are fabricated in a shape memory material such as copper-based, NiTi (nickel and titanium)-based, and/or polymer shape memory materials. By fabricating each of the first and second jaws in such a memory material which may deform but which will return to its normal state the jaws can be configured through a suitable shaping of the jaws to be naturally biased towards one another. In this way the jaws may be separated to allow for the location of an organ or other tissue therebetween. The construct of the jaws is such that jaws will tend to move towards one another, the movement effecting a capture or clasping of the organ/tissue therebetween in a similar fashion to that described heretofore with the grasper configurations of FIGS. 1 through 18. In this arrangement however, the jaws are spaced apart from another, an upper jaw comprising a kink, the kink effecting a biasing of the upper jaw towards the lower jaw such that the jaws are naturally biased towards one another. Similarly to the previously described devices, this clasping device is also configured such that the jaws are operably separable to allow for the location of an organ or other tissue therebetween. However in these arrangements the first jaw 2115 comprises first 2140 and second 2141 segments angularly offset from one another. In the examples shown the first segment extends away from the second jaw 2116 and the second segments 2141 extends towards the second jaw 2116. The first jaw may comprise a third segment 2142, provided at an end portion of the second segment 2141, the third portion defining the tip of the first jaw. This tip may be provided in atraumatic or traumatic configurations.

In the configurations of FIGS. 21C through 21G, the third segment comprises a planar portion extending from the second segment, the planar portion being substantially parallel with the second jaw 2116. In the arrangements of FIGS. 21A to 21D and 21G to 21H, the third segment comprises a curved surface, the curved surface defining the end of the first jaw. Where the third segment comprises both a curved surface and a planar portion, the planar portion is provided between the curved surface and the second segment.

The device of FIG. 21 is desirably collapsible upon itself such that the upper arm 2115 is brought into intimate contact with the lower arm 2116. This is affected by applying a force at the shoulder 2150 to effect a movement of the tip 2142 of the upper arm in a direction downwardly towards the corresponding end 2143 of the lower arm 2116. In a collapsed configuration the first and second arms are substantially parallel to allow for the insertion of the device through a narrow bore trocar or cannula. By fabricating in a memory material, on removal of the force the arms will tend to separate and adopt their normal configuration—as shown in FIG. 21.

Such a clasping element may be used in combination with a suture or other fastening means to effect a movement of the clasped organ or tissue from its normal resting position to an operational site where, for example, access to a site normally occluded by the normal resting position is required. For example as shown in FIGS. 21C through 21G one or more suture apertures 2110 may be provided in a back portion 2125 of the device to provide for a coupling of the device to a suture. Multiple suture tie holes such as shown in FIG. 21G provide the benefit of increased flexibility over this area. Radiused or chamfered curved edges may be incorporated for less traumatic applications. The suture may be supplied pre tied to the element. Various iterations featuring teeth on lower 2136 and/or upper 2135 jaws are shown above. Features included enable easier removal of the device with tabs for suture tying. The teeth may be traumatic or atraumatic. The biasing force of the arms can be predefined to adjust a distance between the two at the mouth 2130 of the device. For example as shown in FIG. 21B, on adoption of the normal closed position, a gap 2130a is provided in the mouth region between the upper 2115b and lower 2116b arms. In contrast in FIG. 21C, the biasing force is such that on adoption of the closed position the gap 2130a is reduced to a negligible amount, the arms are in intimate contact with one another in the mouth 2130 region.

The force is a factor of the design of the angles used to separate the upper 2135 arm from the lower 2136 arm. This angle, theta, shown in FIGS. 21A, 21B and 21C has a radius defining the separation between the upwardly 2140 and downwardly 2141 segments of the upper arm 2135. This angle separating the first segment from the second segment is desirably an acute interior angle. The angle phi offsetting the first segment from the second jaw 2116 is also desirably an acute interior angle. In this way, the first and second segments of the first jaw and a portion of the second jaw are arranged relative to one another to define an acute triangle. All radii can be adjusted to achieve more or less closure force depending on the application.

As shown in FIGS. 21A through 21D and FIGS. 21F through 21H, the second jaw may be configured to extend further than the first jaw, such that on operable presentation of the device to an organ or tissue, the second jaw will abut against the organ or tissue prior to the first jaw.

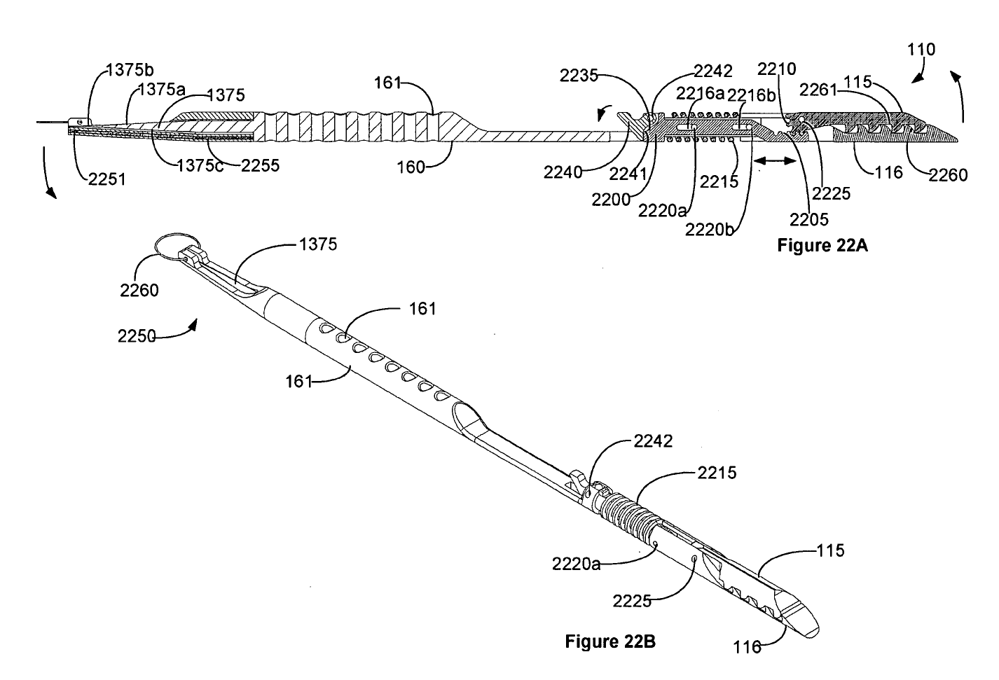

FIG. 22 shows a further example of a grasper in accordance with the present teaching. This grasper employs a different arrangement for effecting an opening of the mouth through separation of the upper 115 and lower 116 jaws. Common with previously described arrangements, an actuator is provided which is moveable in a direction substantially transverse to the direction of opening of the jaw. In this arrangement it may be considered as being moveable in a direction substantially parallel with the longitudinal axis of the device. In this arrangement the actuator comprises a moveable member 2200 which is coupled via interlocking teeth 2205, 2210 provided on each of the moveable member 2200 and the upper jaw 115. Movement of the actuator in a linear fashion effects, through the gearing provided by the interlocking teeth 2205, 2210, a rotational movement of the upper jaw away from the lower jaw 116 to provide for an opening of the mouth. The upper jaw 115 is pivotable about a pivot point 2225 which in combination with the interplay between the interlocking teeth controls the rotational movement.

The moveable member 2200 is coupled to a biasing means—in this exemplary arrangement provided by a spring 2215 that acts to normally retract the moveable member back towards the main body portion 160 of the grasper and away from the mouth 110. In this way the moveable member will, in the absence of an external force acting thereon effect a closure of the upper jaw 115 relative to the lower jaw 116 so as to provide the mouth in a normally closed configuration. The spring 2215 in this exemplary arrangement extends circumferentially about the moveable member 2200 along a substantial length of the moveable member 2200. Springs may be loaded in series in this embodiment to facilitate a greater closure force; in this case each spring would be placed within the assembly so that each is separately loaded to ensure optimum translation of their compressive force to closure of the jaw.

The linear movement of the actuator may be controlled by providing first and second apertures 2216a, 2216b on side walls of the moveable member 2200 and coupling two pins 2220a, 2220b into said apertures. The length of the apertures define the maximum distance of travel of the moveable member and by coupling it in at least first and second positions maintain the movement in a linear fashion. It is desirable that this travel of the moveable member be controlled such that it does not travel fully to meet with and contact the lower jaw 116.

Movement of the moveable member is effected through application of an external force thereon. In this exemplary arrangement, the moveable member comprises a curved end surface 2235 that is co-operable with a curved surface 2241 provided on an actuator handle 2240. Rotation of the actuator handle relative to a pivot point 2242 and in this exemplary arrangement in a downward direction towards the body 160 of the grasper, effects, through the interplay between the curved surfaces 2241, 2235 of the handle 2240 and the moveable member 2200, a translation of this rotation motion into the linear motion of the moveable member. At the other end of the moveable member the interplay between the interlocking teeth 2205, 2210 effects a further translation of the linear motion to a rotational motion of the jaws relative to one another. It will be appreciated that the combination of the handle, moveable member and teeth provided on each of the member and the upper jaw 115 are exemplary of a configuration that allows such translational between a rotational driving force to a linear actuating force to a rotational opening of the jaws. In this way it may be considered a cam arrangement whereby the cam surfaces and follower effect translation of circular to reciprocating to circular motion.

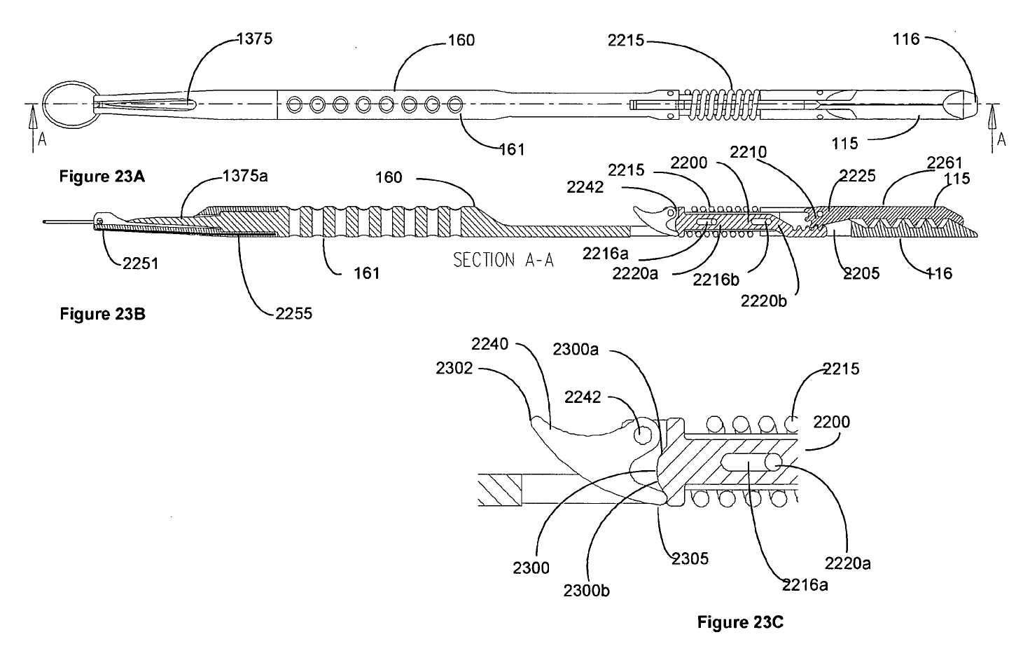

In this embodiment, rearward facing teeth 2260 are mated with flat or planar surfaces 2261 on the opposing jaw. This feature provides two benefits: a space is created between each set of teeth to accommodate tissue during grasping and the rearward sloping direction of the teeth improves tissue retention once grasped. FIG. 23 provides the same spacing or gap between the received teeth in the opposing jaw, but the teeth are not rearward facing.

FIG. 23 shows a further example of how an actuator may be employed to effect translation of circular to reciprocating to circular motion. In this arrangement the coupling between each of the moveable member 2200 and the upper jaw 115 is as described with reference to FIG. 22. A difference is at the coupling interface between the moveable member 2200 and the actuating handle 2240. In the arrangement of FIG. 22, the curved surface 2241 of the moveable member presented one curved element, in the form of a shoulder, to the surface of the handle. The handle moves through an arc and the movement through the arc determines the length of travel of the moveable member. In the arrangement of FIG. 23, the end region of the moveable member defines a nipple 2300 projecting substantially perpendicularly to the direction of movement of the moveable member. The nipple comprises first 2300a and second 2300b curved surfaces which meet at a mid-point of the nipple. Movement of an end portion 2302 of the handle downwardly about a pin 2242 effects a corresponding movement of an abutment surface 2305 along the nipple 2300 to effect the linear motion of the moveable member towards the mouth region of the grasper device. Similarly to that described with reference to FIG. 22, the removal of the external force acting on the handle 2240 allows the biasing action of the spring 2215 on the moveable member to dominate effecting a return of the moveable member to its normal position, with the jaws closed relative to one another. It will be appreciated that the nipple arrangement is one example of how the interaction between the actuator handle and the moveable member may be extended to effect subsequent extension in the length of travel of the moveable member.

The arrangements of FIGS. 22 and 23 also include modifications to the distal end of the device, i.e. that end of the device that is furthest from the mouth region. It will be recalled from, for example, FIGS. 12 and 13 that certain configurations of a grasper in accordance with the present teaching employ a deployable needle 1275, 1375 which is normally provided in a sheathed arrangement to prevent inadvertent contact with abdominal features but which when deployed allows a fixing of the grasper to an inner abdominal wall or other desired feature. The configurations of FIGS. 22 and 23 also employ such a deployable needle 1375. In these exemplary arrangements, an upper surface 1375a of the needle is always visible but the tip 1375b and lower surfaces 1375c are protected in the sheathed or shielded configuration by their proximity to a barrier 2250 that is defined within an end region 2251 of the grasper. In these exemplary arrangements the barrier 2250 is provided by a flexible material which can be flexed downwardly away from the upper surface 1375a of the needle to expose the tip 1375b and lower surface 1375c of the needle 1375. Once exposed, the needle can be embedded into the abdominal wall or other feature to secure the grasper as desired.

To ensure that the flexible material does not inadvertently move away from the needle, it may comprise a strengthening member or rib, 2255, which will act as a spring to return the flexible material to its original position. This rib may be fabricated from a shape memory material, such as Nitinol, stainless steel wire, or polymer rod. By having such a material it is possible to ensure that the flexible material will normally adopt its sheathed position relative to the needle but which on application of a force thereon may be moved away from the needle allow deployment. The flexible material may also be fabricated from a material having a durometer value sufficient to allow it to return automatically within its elastic limit.

The graspers may employ a hook or other such coupling member 2260 provided at the end region 2250 to allow coupling of an external member or device to the grasper. This may be used to effect a deployment of the needle or simply to allow for a controlled transport of the grasper itself within for example the abdominal cavity and removal via a trocar or cannula at the end of the procedure

FIG. 24 shows a further example of how an actuator may be employed to effect translation of reciprocating or linear motion to circular motion. In this arrangement the coupling between the moveable member 2200 and the upper jaw 115 is as described with reference to FIGS. 22 and 23. A difference is in how the moveable member is driven. In the arrangements of FIGS. 22 and 23, a coupling interface between the moveable member 2200 and a separate actuating handle 2240 was provided. In this arrangement of FIG. 24, the moveable member 2200 may be directly driven by action of an external driver thereon. The moveable member comprises a coupling surface 2400 which can be used to engage with the moveable member. Action on the moveable member is used to effect a sliding or linear motion of the moveable member with the device, that sliding being translated by the inter-engagement of the teeth 2205, 2210 of each of the moveable member and upper jaw 115. Again, the action of the biasing spring 2215 will cause the moveable member to normally move towards the needle 1375 end of the device effecting a closure of the two jaws relative to one another.

As is clear from examination of each of FIGS. 22 to 24, the teeth of this configuration are substantially out of phase with one another with the teeth of the upper jaw being received into recesses formed between the teeth of the lower jaw and vice versa. In this configuration the teeth 2435, 2436 are narrower than the recesses so that gaps are provided between the recess walls and the teeth received therein on closure of the jaws relative to one another. The jaws 115, 116 are not provided of the same length so as to present a chamfered end surface at the end region of the device.

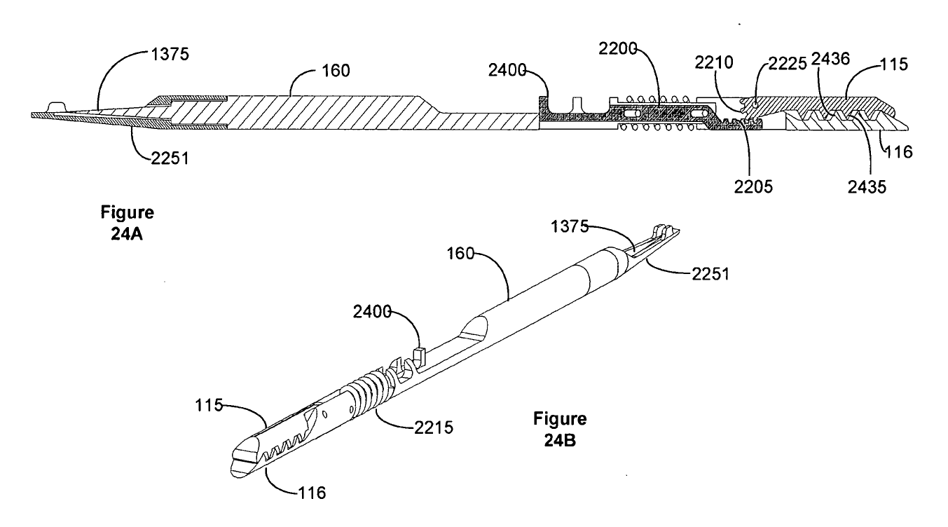

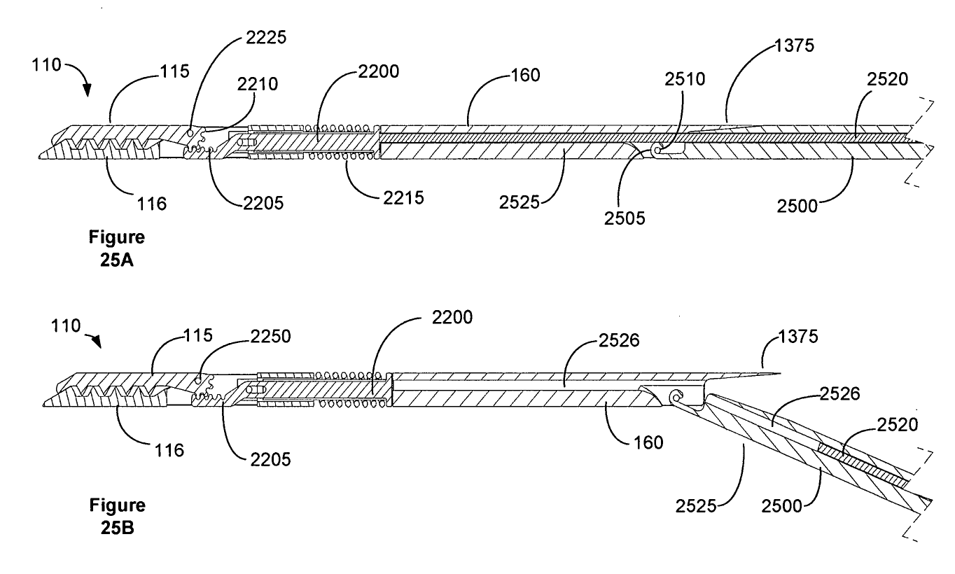

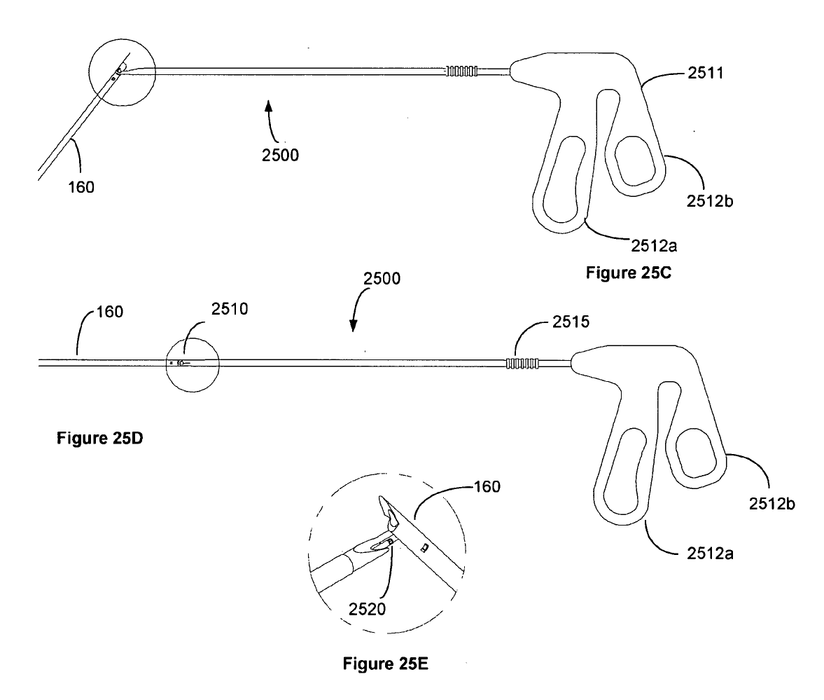

FIG. 25 shows a modification to the arrangement of FIG. 24 whereby the movement of the jaws away from one another is again provided by a translation of linear to rotational motion, but in this instance the linear motion of the moveable member 2200 is provided by action of an external actuator 2500 that is coupled at an end region of the grasper. Engagement of the two is provided by a hook 2505 and pin 2510 coupling which allows for the angular presentation of the external actuator 2500 to the grasper (FIG. 25B, FIG. 25C) and then rotation of the actuator 2500 relative to the grasper to achieve linear alignment (FIG. 25A, FIG. 25D). The external actuator 2500 may comprise a handle 2511 having a pair of finger grips 2512a, 2512b. Movement of the finger grips 2512 allows for a corresponding movement of a driver 2520 within a shaft 2525 of the actuator 2500. Once linear alignment of the actuator 2500 to the grasper has been achieved this movement of the driver 2520 through the shaft 2525 can be used to effect the linear motion of the moveable member 2200, through an extension of the driver 2520 through a corresponding shaft 2526 of the grasper and into contact with the moveable member 2200.

The material used to fabricate such a device may be metallic or plastic shape memory, metal, plastic, ceramic or composites thereof. The whole suture/clasp assembly may be made from a bio-degradable material and left in situ for medium term retraction. The assembly can be made with two clasps connected by suture for short or medium term retraction. The assembly may be made from a biocompatible material such as a thermoplastic polyurethane or metallic alloy enabling permanent implantation. It will be appreciated that as this clamping arrangement is configured for complete deployment within the abdominal cavity and will tend to a normally closed position which on deployment will effect a clasping of the organ or tissue between the arms until actively released. For this reason such a clasp could be deployed for short term, medium term and permanent implant applications. While not intending to limit the grasper or clasp assembly of the present teaching to any one specific set of parameters or materials it will be appreciated that for use in deployment through a trocar that such a device should have an outside diameter smaller than the bore of the trocar to allow complete passage of the device through the trocar. The length of the grasper of all figures bar that of the arrangement of FIG. 11, where the grasper is coupled to a suture, is desirably of the order of 150 mm with the length of the mouth being greater than about 15 mm and desirably less than about 30 mm. The device could be fabricated in any one of a plurality of different materials, examples of which are PEEK, ceramics, metals, plastics, or composites thereof. The jaws could be symmetric with one another or could be asymmetric. The spring may be of metallic or polymer construction. A shape memory spring material would be useful in embodiments which employ springs disposed between the jaws 115 and 116 to enable a larger opening without permanent deformation occurring. The spring component may also be placed in series (longitudinally) or in parallel (vertically) to achieve greater closure forces.

While preferred arrangements have been described in an effort to assist in an understanding of the teaching of the present invention it will be appreciated that it is not intended to limit the present teaching to that described and modifications can be made without departing from the scope of the invention.

Where features or elements of a surgical device have been described with reference to one figure or arrangement it will be appreciated and understood that such features or elements could be interchanged with or replaced by features or elements of another figure or element. It will therefore be understood that components described with reference to one example of a surgical device in accordance with the teaching of the present invention are not to be construed as being limited exclusively for that example. In this way there are many modifications that can be made to the exemplary embodiments described herein.

The words comprises/comprising when used in this specification are to specify the presence of stated features, integers, steps or components but does not preclude the presence or addition of one or more other features, integers, steps, components or groups thereof.

1. A laparoscopic surgical device comprising a body coupled to a head, the head comprising a pair of jaws defining a mouth within which at least a portion of an organ or tissue may be grasped, the device being dimensioned to be operably passed fully through a trocar into an abdominal cavity wherein it may be manipulated by a surgeon or other operator to grasp a desired target organ or tissue, the device further comprising an actuator coupled to at least one of the first and second jaws, operable application of a force onto the actuator effecting a movement of the actuator within the device providing a corresponding opening of the mouth of the device through a translation of linear motion of the actuator to a rotational motion of the jaws relative to one another.

2. The device of claim 1 comprising a spring or other biasing means configured to provide an operable biasing of the jaws towards one another, the jaws being biased towards one another so as to normally adopt a closed configuration.

3. The device as claimed in claim 1 configured such that operably on grasping at least a portion of the organ or tissue within the mouth of the device, the jaws are naturally biased towards one another so as to retain the portion of the organ or tissue, until application of a second force action on the actuator to effect an opening of the jaw, such that movement of the device will effect a corresponding movement of the retained organ.

4. (canceled)

5. The device of claim 1 wherein the actuator is moveable in a direction substantially transverse to the direction of opening of the jaws.

6. The device of claim 1 wherein the actuator is moveable in a direction substantially parallel with the longitudinal axis of the device.

7. The device of claim 1 wherein the actuator comprises a moveable member which is coupled via interlocking teeth provided on each of the moveable member and one of the first and second jaws.

8. The device of claim 7 wherein operably movement of the actuator in a linear fashion effects, through the gearing provided by the interlocking teeth a rotational movement of the first jaw away from the second jaw to provide for an opening of the mouth.

9. The device of claim 8 wherein the first jaw is pivotable about a pivot point which in combination with interplay between the interlocking teeth controls the rotational movement of the jaws.

10. The device of claim 7 wherein the moveable member comprises first and second apertures, the device further comprising first and second pins engageable with the first and second apertures, wherein the length of the apertures define the maximum distance of travel of the moveable member.

11. (canceled)

12. The device of claim 7 wherein the moveable member is co-operable with an actuator handle, rotation of the actuator handle relative to a pivot point effecting, through an interplay between the handle and the moveable member, a translation of rotation motion into the linear motion of the moveable member.

13. The device of claim 12 wherein the curved end surface is provided at an opposing end of the moveable member to the interlocking teeth.

14. The device of claim 12 wherein the combination of the handle, moveable member and teeth provided on each of the moveable member and the jaw provide a translation between a rotational driving force to a linear actuating force to a rotational opening of the jaws.

15. The device of claim 1 wherein the first jaw comprises teeth which are mateable with flat or planar surfaces on the opposing jaw.

16. The device of claim 7 wherein the moveable member comprises a curved surface which is co-operable with a handle surface, movement of the handle through an arc effecting a corresponding movement of and determines the length of movement of the moveable member.

17. The device of claim 16 wherein the moveable member defines a nipple projecting substantially perpendicularly to the direction of movement of the moveable member.

18. The device of claim 17 wherein the nipple comprises first and second curved surfaces which meet at a mid-point of the nipple such that movement of an end portion of the handle downwardly about a pin effects a corresponding movement of an abutment surface along the nipple to effect the linear motion of the moveable member towards the mouth region of the grasper device.

19. The device of claim 7 wherein the moveable member comprises a coupling surface for use with an external driver, such that on operable engagement of the driver with the coupling surface, a driving force applied by the driver is useable to effect a sliding or linear motion of the moveable member within the device, that sliding being translated by the inter-engagement of the teeth of each of the moveable member and the first jaw to a movement of the jaws relative to one another.

20. The device of claim 19 wherein the coupling surface comprises a pin which is co-operable with a hook of an external driver, on engagement of the driver with the coupling surface the driver is moveable within a shaft of the actuator.

21. The device of claim 1 wherein at least one of the jaws is pivotable about a pivot point relative to the other of the jaws to allow for movement of the jaws away from one another to facilitate the presentation of an organ into the mouth of the device.

22. The device of claim 21 wherein at least a portion of the actuator defines a contact surface for allowing provision of an external force so as to effect a separation of the jaws away from one another, the device further comprising a channel defined in an upper surface of the body portion to provide access to the contact surface.

23. The device of claim 21 comprising a pivot pin located at the pivot point and coupling the jaws to one another.

24. The device of claim 23 wherein the device comprises a longitudinal axis, the pin being mounted so as to be substantially perpendicular to the longitudinal axis.

25. The device of claim 23 wherein the pivot pin provides a fulcrum such that application of a force to a first side of the pin effects a corresponding movement of the jaws on a second opposing side of the pin.

26. The device of claim 25 configured such that application of a force to a first side of the fulcrum effects a movement of that first jaw away from the second jaw.

27. The device of claim 25 configured such that application of a force to a first side of the fulcrum effects application of a force to each of the first and second jaws such that the separation of the jaws from one another is effected by a relative movement of each of the first and second jaws.

28. The device of claim 23 comprising a spring located between the pivot pin and the mouth of the device.

29. (canceled)

30. The device of claim 1 wherein at least one of the jaws comprises teeth.

31.-32. (canceled)

33. The device of claim 30 wherein each of the first and second jaws comprise teeth and the teeth of the first and second jaws are configured such that on bringing the first and second jaws towards one another, the teeth of the first jaw will inter-engage with the teeth of the second jaw so as to allow a complete closing of the mouth of the device.

34.-35. (canceled)

36. The device of claim 1 wherein at least one of the jaws comprises a tapered lead-in portion, tapering from a distal portion of the jaws inwardly towards the mouth such that, in a closed orientation, the jaws are separated from one another at the lead-in portion.

37.-38. (canceled)

39. The device of claim 1 wherein the body portion is coupled to or integrally formed with at least one of the jaws.

40. (canceled)

41. The device of claim 39 wherein the body portion provides a coupling contact for effecting a securing of the device at a desired position within the abdominal cavity.

42.-43. (canceled)

44. The device of claim 1 comprising a plurality of coupling surfaces spaced apart along the body portion and wherein the coupling surfaces are provided as a plurality of individual coupling ports extending through the body portion.

45.-48. (canceled)

49. The device of claim 39 wherein the body is configured to be moveable relative to the jaws of the device.

50.-61. (canceled)

62. The device of claim 1 wherein the body portion is segmented to comprise a head proximal body portion and a head distal body portion, the head proximal portion being adjacent to the jaws of the device and the head proximal body portion and head distal body portion of the body portion are moveable relative to one another.

63.-64. (canceled)

65. The device of claim 1 wherein the body portion comprises a deployable needle or clamp so as to allow a fixing of the device to an inner abdominal wall or other desired feature.

66.-77. (canceled)

78. The device of claim 1 wherein the first and second jaws are coupled to one another and each are moveable relative to the other to adopt the open position, the device further comprising a linkage between the actuator and each of the first and second jaws.

79.-103. (canceled)

104. A coupling tool for use with a device as claimed claim 1, the tool comprising:
a. a needle portion for operably passing through an abdominal wall from an exterior portion of a body to an interior portion of the body;
b. a contact surface for operably resting against an exterior portion of the body, and
wherein operably the tool is configured to engage with and maintain the device in a predetermined position within the abdominal cavity.

105. The tool of claim 104 wherein the needle portion is configured to engage with the device, operable inter-engagement of the needle portion with the device effecting a securing of the device against the exterior portion of the body through contact of the contact surface against the body.

106. The tool of claim 104 wherein the contact surface is textured and/or provided with an adhesive to improve retention of the tool against the body.

107. The tool of claim 104 wherein a tip region of the needle comprises one or more blades.

108.-115. (canceled)

116. A method of moving an organ or tissue within an abdominal cavity during laparoscopic surgery, the method comprising:
providing a device as claimed in claim 1;
disposing that device through a trocar or cannula into the abdominal cavity;
biasing the jaws open to receive the organ or tissue and then removing the biasing force such that the jaws grasp the organ or tissue within the mouth of the device;
moving the device to effect a corresponding movement of the grasped organ or tissue;
anchoring the device to maintain the attained position.
completing the required surgery;
releasing the device; and
removing the device from the abdominal cavity through a trocar or cannula.
